(19) United States
(12) Patent Application Publication
(10) Pub. No.: US 2003/0003449 A1
(43) Pub. Date: Jan. 2, 2003

(54) METHODS AND COMPOSITIONS FOR THE DETERMINATION OF PROTEIN FUNCTION AND IDENTIFICATION OF MODULATORS THEREOF

(76) Inventors: ROLF MENZEL, YARDLEY, PA (US); VLADIMIR KHAZAK, PRINCETON, NJ (US)

Correspondence Address: PENNIE AND EDMONDS, 1155 AVENUE OF THE AMERICAS, NEW YORK, NY, 100362711

(21) Appl. No.: 09/359,595

(22) Filed: Jul. 23, 1999

Related U.S. Application Data
(60) Provisional application No. 60/093,855, filed on Jul. 23, 1998.

(51) Int. Cl. C12Q 1/68

(52) U.S. Cl. 435/6

(57) ABSTRACT

The present invention provides libraries of tag dominant-negative elements (TDNE) and methods the identification specific TDNEs that act as dominant-negative elements on a target protein of interest. The present invention further relates to the use of such TDNEs and dominant-negative elements for the identification of protein-protein interactions, and the determination of a target protein's biological activity and function. Furthermore, the present invention relates to the development of means, including small molecule compounds, for disrupting the target protein's biological function and activity.

This application claims priority under 35 U.S.C. § 119(e) to provisional patent application no. 60/093,855, filed Jul. 23, 1998, the entire contents of which is incorporated herein by reference in its entirety.

### 1. FIELD OF THE INVENTION

The present invention relates to methods and compositions for the identification of fusion proteins that can act as dominant-negative modulators of particular protein interactions. The present invention further relates to the use of such fusion proteins for the identification of amino acid sequences responsible for particular protein-protein interactions, and the determination of the biological activity and function of a target protein involved in such protein-protein interactions. Furthermore, the present invention relates to screening assays for identifying compounds, including small molecule compounds, that modulate, e.g., disrupt, the protein-protein interactions and can, therefore, modulate, e.g., disrupt, the target protein's biological function and activity. The present invention still further relates to methods and compositions for modulating, e.g., disrupting, the protein- protein interactions and, therefore, the target protein's biological activity.

### 2. BACKGROUND OF THE INVENTION

General Background. In the past decade it has become apparent that many diseases result from genetic alterations and from over- or under-representation of essential gene products. Such diseases include conditions related to unregulated cell proliferation, such as cancer (Halaban, 1996, Semin. Oncol. 23:673-681; Russell et al., 1995, Leuk. Lymphona 16:223-229), atherosclerosis (Libby et al., 1997, Int. J. Cardiol. 62 Suppl. 2:S23-S29; Schachter, 1997, Int. J. Cardiol. 62 Suppl 2:S3-S7; Ruschitzka et al., 1997, Cardiology 88 Suppl 3:3-19) and psoriasis (Gniadeck, 1998, Gen. Pharmacol. 30:619-622; Van de Kerkhof and Van Erp, 1996, Skin Pharmacol. 9:343-354), as well as inflammatory conditions such as rheumatoid arthritis (Grimbacher et al., 1998, Arthritis Rheumatol. Int. 17:185-192; Jackson et al., 1997, FASEB J. 11:457-465), and metabolic diseases such as diabetes mellitus (Blom et al., 1993, Md. Med. J. 42:549-556). The finding that many of these diseases are based on aberrant protein expression has refocused the medical community to seek new modalities for disease management which includes the designing of drugs which directly act on the accountable target gene or protein. In order to develop highly specific drugs which directly interfere with a target gene or protein, the target gene or protein accountable for a specific disease first needs to be identified.

Recently, developments in genomic sciences have tremendously accelerated the cloning of new genes and the identification of their respective nucleic acid sequences. One major problem researchers are facing today is the assignment of functions to the numerous newly discovered genes and their gene products, and identification of their interactions with other components inside or outside a cell. The nucleotide sequence may be revealing if it leads to identification of a protein product which is homologous to a family which had been characterized previously, e.g., the family of tyrosine kinases (Gullick, 1998, Biochem. Soc. Symp. 63:193-198; Haque and Williams, 1998, Semin. Oncol. 25 (1 Suppl. 1): 14-22; O-Shea et al., 1997, J. Clin. Immunol. 17:431-44), proteases (Estrov and Talpaz, 1996, Cytokines Mol. Ther. 2:1-11; Cawston, 1995, Br. Med. Bulls. 51:385-401; Powers et al., 1993, Agents Actions Supl. 42:3-18), transcription factors and other DNA binding proteins (Ogbourne and Antalis, 1998, Biochem. J. 331:1-14; Lefstin and Yamamoto, 1998, Nature 392:885-888; Tan and Richmond, 1998, Curr. Opin. Struct. Biol. 8:41-48), etc. In general, however, the problem of identifying the function of the gene, i.e., what the encoded protein actually does in a living cell, will remain.

The classical approach for identifying the function of a protein of interest has been to inactivate the gene and characterize the effects of such inactivation. A number of different approaches have been employed to inactivate a gene or to disrupt the function of its product. These approaches have been reviewed by Herskowitz, 1987, Nature 329:219-222, and are summarized below.

Strategies Presently Employed For The Inactivation Of Genes Or Their Products. One technique which has proven to be a powerful approach for the elucidation of a gene's and its product's function is gene disruption by means of homologous recombination. One advantage of the technique of gene disruption is that the gene function is completely destroyed, leading to very clear results. However, this technique is only efficient and easily applicable to yeasts (Scherer and Davis, 1979, Proc. Natl. Acad. Sci. U.S.A. 76:4951-4955; Rothstein, 1983, Meth. Enzym. 101: 202-211; Russell and Nurse, 1986, Cell 45:145-153), Aspergillus (Miller et al., 1985, Molec. Cell. Biol. 5:1714-1721; May et al., 1985, Molecular Genetics of Filamentous Fungi (ed. Timberlake, W. E.), pp. 239-251) and Dictyostelium (De Lozanne and Spudich, 1987, Science 236:1086-1091). In higher eukaryotes, DNA segments can be added to the genome in random positions, however, inactivation of the equivalent wild-type gene by targeted homologous recombination is a rare event. Thus, the generation of higher organisms carrying a desired gene disruption is a very time- and labor-intensive and expensive approach. A number of techniques and strategies have been described for the generation of so-called knock-out animals, mostly mice (see, e.g., Capecchi, U.S. Pat. No. 5,631,153; and Thomas et al., 1992, Mol. Cell. Biol. 12:2919-2923). The phenotype of such animals has in a number of cases revealed interesting insight in the function of genes and their products. James et al, 1998, Circ. Res. 82:407-415; Gingrich and Roder, 1998, Annu. Rev. Neurosci. 21:377-405; Murakami and Honjo, 1997, Curr. Opin. Immunol. 9:846-850. However, the total time required to obtain a “knock-out” animal is still very long.

An alternative approach that has attracted attention in recent years involves the use of antisense nucleic acids to block the expression of a gene by preventing the translation of its transcript. Izant and Weintraub, 1984, Cell 36:1007-1015; Melton, 1985, Proc. Natl. Acad. Sci. U.S.A. 82:144-148. This approach has been successfully employed in a number of examples and has assisted in shedding light on the function of several genes and their products in a number of organisms, including mammals. North, 1985, Nature 313:635. For example, a phenocopy of Drosophila Kruppel mutants has been produced by injecting antisense Krüppel RNA into wild-type embryos. Rosenberg et al., 1985, Nature 313:703-706. Furthermore, a discoidin-deficient slime-mould has been generated by expressing antisense discoidin oligonucleotides in vivo (Crowley et al. 1985, Cell 43:633-641), and in murine NIH 3T3 cells, antisense oligonucleotides of the proto-oncogene c-fos have been demonstrated to inhibit the platelet-derived growth factor (PDGF)-induced growth (Nishikura and Murray, 1987, Mol. Cell. Biol. 7:639-649). However, the antisense approach can be very labor-intensive, requiring identification of suitable regions in the mRNA which are accessible to antisense oligonucleotides. Further, this approach may, in certain cases, fail to ultimately reveal the sought-after phenotype of the gene of interest.

Others have reported the creation of a mutant phenotype by overexpression of a wild-type gene (Meeks-Wagner and Hartwell, 1986, Cell, 44:43-52). The rationale of this approach is that an imbalance of subunit concentration of protein complexes can have severe consequences to the proper formation of multi-protein structures, such as the cytoskeleton and the histone scaffolding of eukaryotic chromosomes. For example, using this approach it has been shown that an altered ratio of histones H2A-H2B to H3-H4 resulting from overexpressing the genes for histones H2A-H2b leads to chromosome loss (Meeks-Wagner and Hartwell, 1986, supra). The inherent limitation of this approach is apparent,—it can only be used for genes whose products are part of multi-protein complexes.

Another approach which has been employed to inhibit gene function involves the use of antibodies against synthetic antigens based on the predicted sequence of a gene product of interest. For example, it has been shown that antibodies against microtubule components can alter the morphology of intermediate filaments (Blose et al., 1984, J. Cell Biol. 98:847-858; Wehland and Willingham, 1983, J. Cell Biol. 97:1476-1490). Antibodies against ras proteins can transiently reverse the transformed phenotype of ras-transformed cells (Feramisco et al., 1985, Nature, 314:639-642; Kung et al., 1986, Expl. Cell Res. 162:363-371). However, although suitable for the analysis of individual cells or even groups of cells, this method is limited to transient perturbation of the wild-type gene function because of antibody dilution and degradation.

About one decade ago, another new strategy for the elucidation of gene function was described which involves blocking of the gene function at the protein level. In this approach, the cloned gene is altered so that it encodes a mutant product capable of inhibiting the wild-type gene product in a cell, thus causing deficiency in the function of that gene product. Herskowitz, 1987, Nature 329:219-222; Hozmeyer et al., 1992, Nucleic Acids Research 20:711-717; Gudkov et al., 1993, Proc. Natl. Acad. Sci. USA 90:3231-3235; U.S. Pat. No. 5,665,550; U.S. Pat. No. 5,217,889. A mutation of this kind, by its nature, is dominant, because its phenotype is manifested in the presence of the wild-type gene. Since such mutants inactivate the wild-type gene's function they are referred to as “dominant-negative” mutants. These kind of mutations have been referred to as “antimorphs”, i.e., “antagonistic mutant genes, having an effect actually contrary to that of the gene from which they were derived”. Miller et al., 1985, Molec. Cell. Biol. 5:1714-1721.

Dominant-Negative Mutants. This approach involves the inhibition of the function of a wild-type gene product by an overproduced inhibitory variant of the same product. Dominant-negative inhibitory variants of a gene may act by one of several mechanisms. First, many, if not the majority of, proteins are functional in multimeric complexes. Thus, in the case of a multimeric protein, a derivative capable of interacting with wild-type polypeptide chains but which is otherwise defective will be inhibitory if it causes the formation of non-functional multimers. For example, receptor tyrosine kinases have been shown to form oligomeric complexes in response to binding of their respective ligand; this oligomerization is necessary for the enzymatic domain to be activated. Accordingly, as shown for a number of members of the receptor tyrosine kinase family, a defective variant of the receptor which is still capable of oligomerization acts as an inhibitory dominant-negative mutant (Kashles et al., 1991, Mol. Cell. Biol. 11:1454-1463; Redemann et al, 1992, Mol. Cell. Biol. 12:491-498; Millauer et al., 1994, Nature 367:576-579).

Furthermore, it may also be possible to form inhibitory variants of monomeric proteins. If, for example, the activity of a protein is limited by the availability of substrate, then a variant capable of binding substrate but not of carrying out a subsequent catalytic step could be inhibitory. Alternatively, dominant-negative elements affecting a monomeric protein could act by blocking proper folding (Michaels et al., 1996, Proc. Natl. Acad. Sci. USA 93:14452-14455).

Although large-scale systems for genetic selection of dominant inhibitors have been attempted, they have had limited success. In one case, for example, a system was developed to identify dominant inhibitors of the yeast pheromone response pathway by expressing DNA libraries encoding genome-derived fragments or random peptides in yeast cells, and either selecting for expression of an appropriate reporter (PCT publication WO 98/39483), or directly selecting for cells that escape a-factor induced cell cycle arrest (Caponigro et al., 1998, Proc. Natl. Acad. Sci. 95: 7508-13). Another method utilizes the expression of DNA libraries encoding peptides in a cell type of interest, followed by screening for candidates that interfere with, or suppress, some phenotype of that cell (U.S. Pat. Nos. 5,217,889 and 5,811,234). Although both these may identify molecules that interrupt some pathway or cellular phenotype, considerable effort is required to identify the phenotype, determine mechanism of action of the candidate peptide, and locate its target in the host cell.

Such previously described methods are limited in that they rely on elements that involve at least three unknown variables: the dominant negative element, the mechanism of interaction of the dominant-negative element with its target, and, the nature of the target itself. As a result, such methods have several serious drawbacks for applications in high-throughput screening of candidate therapeutic compounds. First, each of these methods requires the identification, expression, and manipulation of a chosen phenotype in a host system, e.g., tissue culture cells in the case of a mammalian system. However, all phenotypes of interest may not be amenable to genetic selection. Moreover, even if genetic analysis is available, slight differences in phenotypic activity may be difficult to detect. Further, high throughput is difficult to achieve in such systems because of the requirement that a particular, possibly different, phentoype be followed for any given protein of interest. Second, peptide fragments expressed in or delivered into a cell may not be large enough or stable enough to provide a sufficient hindrance to the target interaction. Finally, even if a dominant-negative activity is identified by one of these methods, its mechanism of inhibition or interaction can be difficult to unravel. Although function may be inferred by the phenotype of a dominant-negative mutant, subsequent analysis is required to determine its mechanism of action. For example, similar phenotypes can result from diverse mechanisms, or alternatively, disruptions within a single pathway may lead to a variety of phenotypes. Such in-depth analysis is laborious and time-consuming, and thus unsuitable for high-throughput screening methods. Thus, the considerable effort and expense necessary to determine the action of a compound limits the usefulness of these methods for identifying drug candidates and the further development of lead compounds for therapeutic intervention.

Therefore, as yet, no efficient, sensitive, and targeted system has been described that can be used for high-throughput identification of specific competitor molecules of functional protein-protein interactions, or for identifying compositions for modulating such interactions.

### 3. SUMMARY OF THE INVENTION

The present invention relates, first, to methods and compositions for the identification of fusion proteins that can act as dominant-negative modulators of particular protein-protein interactions. Specifically, this invention relates, first, to the generation of large collections of protein fragments, e.g., fusion proteins comprising fragments of a target protein of interest, fused to a carrier “tagged” protein, the use of microbial systems to genetically prescreen such collections for members that demonstrate or disrupt particular protein-protein interactions, and to the fusion proteins themselves. The demonstration of a protein-protein interaction and/or the disruption of a protein-protein interaction is, for example, a means of identifying portions of the target protein involved in the protein-protein interaction of interest and for identifying and enriching for potential dominant-negative forms of the target protein.

The present invention is based, in part, on the development of methods that use fragments of a target protein expressed as fusion or ‘tagged’ protein elements, herein called tagged dominant-negative elements, or TDNEs, to specifically alter the formation of protein-protein interactions. The invention has several advantages over the previously reported methods for identification of dominant-negative suppressor elements. For example, since the elements in the methods described herein are screened for their ability to interrupt specific protein-protein interactions, and since tagged dominant-negative elements may be derived from the gene encoding the target protein itself, the mechanism of action of the dominant-negative element is predetermined by the experimental design, i.e., the dominant-negative inhibitory element will act by competitively inhibiting protein-protein interactions. Second, the tag portion of the element can increase the inhibitory effect of the dominant-negative peptide and thereby increase the sensitivity of the methods described herein, by, for example, stabilizing the peptide fragment and/or providing steric hindrance, as demonstrated in the working examples presented below. The tag portion of the element can also provide additional functionality, such as facilitating manipulation, recovery or detection of the tagged dominant negative element. Finally, in the methods described herein, a single, well-defined phenotype (e.g., reporter gene expression) is used in microbial systems to screen for elements and candidate compounds that target protein-protein interactions. A clearly defined and quantifiable phenotype applicable to a broad spectrum of proteins and adaptable to high-throughput methods for screening for small molecule therapeutics is a distinct advantage over previously described methods that relied on identification, selection or screening of a variety of endogenous phenotypes.

The present invention further relates to methods and assays for characterizing the biological function and activity of target proteins. In particular, because the protein-protein interactions of interest are required for normal function of the target protein in its native cell, the dominant-negative elements in turn can be used as part of methods that serve to identify or further elucidate the function of the corresponding target protein in its native environment and its potential involvement in the development and manifestation of pathological conditions. Thus, the present invention provides a systematic and rapid means to use the information provided by genome sequencing projects to identify the functions of known and newly discovered genes and their products.

The present invention still further relates to identifying compounds, e.g., small molecule compounds, which block particular protein-protein interactions of interest. In particular, such methods utilize the microbial systems of the invention to identify compounds that modulate, e.g., disrupt, interactions between a peptide fusion protein and the “partner” protein with which it specifically interacts. Thus, the present invention allows rapid and high-throughput identification of compounds that can be tested for an ability to act as target-specific therapeutic compounds.

The present invention further relates to methods and compositions for modulating, e.g., disrupting or inhibiting, particular protein-protein interactions, and therefore modulating the biological function and activity of a target protein involved in the protein-protein interaction. Still further, the present invention provides methods for identifying and designing useful modulating, e.g., therapeutic, compounds based on the identification of dominant-negative elements. The present invention further relates to the development of cytological, diagnostic and therapeutic reagents based on the above fusion proteins and knowledge derived from their characterization in both the microbial models and the dominant-negative analysis.

### 3.1. Definitions

Terms used herein are in general as typically used in the art. The following terms are intended to have the following general meanings as they are used herein:

The term Tag Dominant-Negative Element (TDNE) as used herein refers to a protein fragment or peptide that affects particular protein-protein interactions (the “dominant-negative element”), fused to a carrier protein (the “tag”). The protein fragment or peptide may be derived from a selected target protein of interest involved in the protein-protein interaction of interest, or it may be derived from any other source. Although the protein fragment can be of any length, e.g., can approach the length of the full-length target protein, typically, the protein fragment has a length of about six (6) to about 500 amino acids, or about six (6) to about 150 amino acids, preferably it has a length of about six (6) to about fifty (50) amino acids, and most preferably it has a length of about six (6) to about thirty (30) amino acids. The carrier protein may exhibit a selected function, e.g., as cytological or purification tag, as DNA binding domain, as transcriptional activator domain, or as microbe-based compound, e.g., small molecule discovery tool, or it may simply serve as stabilizer against degradation or to promote steric hindrance.

The methods described herein utilize methods and compositions for identifying and modulating protein-protein interactions (either homotypic or heterotypic protein-protein interactions). For purposes of simplicity and clarity of description, one protein involved in the protein-protein interaction of interest is referred to herein as a “target protein” and a second protein involved in the protein-protein interaction of interest is referred to herein as a “partner protein.” It will be understood that the term “target protein” can be considered interchangeable with the term “partner protein” for the purposes of the methods and compositions described herein. It is also to be understood that the terms can refer to the full-length proteins involved in the protein-protein interactions, or to portions thereof that still exhibit the protein-protein interactions of interest. For example, a TDNE library can be used in the methods hereinbelow to identify and characterize dominant-negative elements that modulate, e.g., disrupt or interfere with, and can be used to identify the function of, either the “target protein” or the “partner protein.” involved in a protein-protein interaction.

It is noted that the terms “peptide”, “polypeptide”, and “protein”, as referred to herein, are used interchangeably.

### 4. BRIEF DESCRIPTION OF THE DRAWINGS

FIGS. 1A-1C depict schematic representations of the AraC based approach for the identification of tagged dominant-negative elements (TDNEs). 1A depicts schematically the organization of AraC. 1B depicts AraC chimera and dependency of lac activity on dimerization of the fused domains. 1C depicts the identification of TDNEs that block dimerization of the araC:araC chimera. FIG. 1A depicts a schematic representation of the modified dual-bait system for the identification and isolation of TDNEs.

FIG. 2A depicts a schematic representation of the modified dual-bait system for the identification and isolation of TDNEs.

FIG. 2B depicts various possible phenotypes associated with TDNE candidates identified with the modified dual-bait system.

FIG. 3 depicts various fusion and reporter constructs for the modified dual-bait assays for the identification and isolation of TDNEs interfering with the Ras:Raf interaction.

FIG. 4 depicts fusion constructs and reporter constructs for the positive selection of blocking fragments.

FIG. 5 depicts schematic diagrams of the AraC fusion plasmids with the wild type leucine zipper and the L19P substitution.

FIG. 6 depicts a plasmid map showing the Ras G12V mutation cloned into the vector pSV-neo.

FIG. 7 depicts a map of the Tet-ON plasmid used in these studies.

FIG. 8A depicts the Tre-Luc reporter plasmid.

FIG. 8B depicts the Fos-Luc reporter plasmid.

FIG. 8B depicts the Zeocin expression plasmid used in co-transformation experiments.

FIG. 9 depicts the levels of β-galactosidase activity found in the indicated SKY48 transformants grown in 2% galactose and 1% Raffinose (part A), and the levels of β-galactosidase activity found in SKY48 transformants grown in 2% galactose and 1% Raffinose and 0.2% Glucose (part B).

FIG. 10 depicts (in the top part) the map of the general plasmid used to form fusions to the CI DNA binding domain used to form the Ras fragment TDNE. In the lower part of the figure the details of the cloning site for plasmids pVJ1,2,3 are shown to illustrate the changes made to accommodate the insertion of random fragments which can be present in any of the three possible reading frames.

FIG. 11 shows the sequence of Ras gene. Underlined in the dashed and dotted segment is the Rfrag that was isolated with the TDNE approach using the nuclease CviJI fragmentation strategy. In bold is shown the RBD fragment described as lying at the Ras:Raf protein interface. The CER fragment previously shown to bind is indicated with the dashed segment. The Rfrag overlaps with pieces known to lie at that Ras:Raf interface.

FIG. 12 depicts (in the top part) the plasmid used to express the CadC::Tnfα chimera. Sequence encoding this chimera is shown in the lower part of the figure.

FIG. 13 depicts (in the top part) the CadBA-Lac activity (Y-axis) that CadC::Tnfα is able to support. When CadC::Tnfα expression is increased by increasing amounts of IPTG (X-axis), CadBA-Lac expression is also increased. The lower part shows that co-expression of TNFα, but not pro-insulin is able to reduce the CadC::Tnfα-driven signal.

FIG. 14 depicts the plasmid constructed to co-express MalE fusion proteins with the CadC-chimera.

FIG. 15A depicts the plasmid pWE84 expressing the MalE fusion with residues C77-T172. The lower part of the figure gives the sequence of this fusion.

FIG. 15B depicts the plasmid pVVE85 expressing the MalE fusion with residues D1-V84. The lower part of the figure gives the sequence of this fusion.

FIG. 15C depicts the plasmid pWE90 expressing the MalE fusion with residues D1-L154. The lower part of the figure gives the sequence of this fusion.

FIG. 16 depicts the levels of CadBA-Lac activity (X-axis) seen in a strain where this interaction signal is driven by the CadC::TnfαR chimera. The figure shows that co-expression of a MalE fusion to TNFαR segments D1-L154 and C77-T172 competes the signal but that the D1 -V84 segment does not.

### 5. DETAILED DESCRIPTION OF THE INVENTION

### 5.1. Overview

The present invention relates to compositions and methods for the identification and isolation of fusion proteins that act as dominant-negative elements, referred to as tag dominant-negative elements (TDNEs). More particularly, the invention relates to assays for the identification and isolation of dominant-negative elements from collections of protein fragments, e.g., protein fragments derived from a target protein involved in a specific protein-protein interaction, that are fused to a carrier protein. Such collections are referred to as candidate TDNEs. The assays employ microbial systems that allow for the genetic prescreen of the collections. The assays employ microbial systems that allow genetic prescreening of the collections of candidate TDNEs for members that affect, e.g., exhibit or disrupt, specific protein-protein interactions. The ability to prescreen protein fragments and chimeric fusion-based protein fragment collections for individual members, in a high throughput microbe-based procedure, dramatically simplifies the process of identifying candidate dominant-negative elements in systems such as a mammallian systems.

One element of the present invention is the generation and use of fusion proteins as collections of candidate TDNEs. Fusion proteins are desirable as a source of candidate dominant-negative elements for a number of reasons. First, the mechanism of a dominant-negative mutant's action typically involves specific protein-protein interactions that block the formation of the naturally occurring protein-protein interactions required for native function. Short protein fragments, however, may lack the ability to effectively act as a steric block. The foreign carrier protein may effectively augment this function, as it may act as a significant steric block once anchored to a target protein. As such, the formation of a family of protein fragments attached to a foreign “carrier” protein generates a more sensitive, “powerful” set of dominant-negative mutants. Secondly, the carrier protein will stabilize protein fragments against degradation and thus allow an array of possible dominant-negative interactions that might not otherwise be observed, particularly in the instance of small protein fragments. Further, the carrier protein may be chosen to add selected desirable functions to the protein fragments and peptides. Such carrier proteins may comprise functional domains and motifs including, but are not limited to, cytological tags, DNA-binding domains, transcriptional activator domains, microbe-based small molecule discovery tools, fluorescent peptides, and cellular localization motifs, such as signal sequences.

The second feature of the present invention is the use of microbial “prescreens” to identify members of the library that a activate specific, well-defined reporter gene phenotypes. Typically, the dominant-negative phenotype arises because the dominant-negative protein, presumably present in excess, blocks a normal interaction that is essential for the function of the protein. To do so, the dominant-negative mutant must itself preserve at least part of the protein-protein interaction potential encoded by a properly folded segment of the native target protein. The number of protein segments of the target with such an interaction potential is a subset of the possible protein fragments and schemes to identify such fragments will enrich for the members of interest. The creation of chimeric libraries of, e.g., fragments of a target protein that interacts with a partner prtoein in a protein-protein interaction of interest, fragments fused to proteins that facilitate the study of such chimera in microbial-based interaction analysis systems, aids in the identification of dominant-negative elements present within these chimeric libraries.

Two primary types of schemes are possible. Such a system could be used to identify or “trap” an interaction between a partner protein and a truncated fragment, fused to the appropriate carrier, derived from a protein previously shown to interact. The isolation of interacting partners in a library of chimera identifies members that define constituent interacting peptides. These chimera identify those members most likely to act as dominant-negative elements; interaction is a prerequisite of a dominant-negative element. A second type of approach will involve “blocking” an existing interaction. Such existing interactions can be based on interactions defined in a microbial, e.g., yeast or bacterial, based interaction system. Chimera defined by their “blocking” properties will have a high probability of being dominant-negative elements in that the ability to block a protein-protein interaction of the target protein, or fragment thereof, will already have been shown. The above approaches also provide a means of assuring that in-frame clones have been identified. Only one out of six inserts of randomly generated, e.g., sheared fragments will be correct: one half will have the correct orientation and one third of these will have the correct reading frame. The identification of a “blocking” or “trap” fragment will, therefore, prescreen a library for correctly oriented and in-frame members that have already demonstrated protein-protein interaction properties with the target or particular protein of interest.

In addition to providing a dramatic enrichment for candidate dominant-negative elements, the general approaches described can also provide other reagents useful in functional genomics based on the same fusion protein libraries. For example, individual chimera can be characterized with respect to both “blocking” and “trap” properties. As discussed above, “blocking” chimeras are very likely to define dominant-negative elements. The inclusion of the additional requirement that candidate dominant-negative elements not only display “blocking” properties but also show the “trap” phenotype eliminate some classes of anomalous chimera. It is also possible to identify chimeras that display a “trap” phenotype, but fail at “blocking”. Such a chimera has the ability to interact with the target protein but fails to block a defined protein-protein interaction. Among such chimera are ones that represent cytological reagents useful in cellular localization and tissue distribution studies of the target or partner protein. Chimera utilized and identified via such created with the proposed schemes are based on a common carrier protein(s) unique to the systems employed. These common carrier proteins “tag” the chimera with sequences that can be used to locate the target protein following binding of the chimera to the target protein in either cell or tissue preparations. Such localization provides important information that, together with other information, such as the nature of a dominant-negative phenotype, defines the function of the target protein.

The microbial procedures used to define interacting protein fragments are based on specific, well defined phenotypes amenable to high-throughput screening techniques. For example, the “trap” phenotypes can involve the transcriptional activation of a reporter gene, whereas, the “blocking” phenotype can involve the interaction-based loss of an existing transcriptional signal assayed, e.g., by reporter gene expression. Thus, specific phenotype can be used to identify compounds, e.g., a small molecules that block the “trap” defining interaction, much in the same way “blocking” chimera are defined and identified.

The schemes disclosed herein have a clear advantage over existing schemes to use interaction systems to discover compounds, e.g., small molecules, that affect protein-protein interactions. As stated above, the targeted interaction is known to define an interaction that has been demonstrated, as part of a systematic procedure, to be vital to the function of the protein of interest. Further, the procedures used to define dominant-negative chimera can define a minimal interacting peptide that will cover a minimal interacting surface. A reduced interaction surface allows the identification of small molecules that do not block larger interacting surfaces, and thus provide molecules of interest that would not be found without deliberately looking for a minimal, but functionally defined, by the dominant-negative phenotype, target in a hybrid interaction screen.

Lastly, the schemes described create chimera that can be screened in microbial systems to identify candidates that should show a dominant-negative phenotype. As discussed above, such dominant-negative chimera can be utilized in defining gene function. The dominant-negative chimera, however, can ablate gene function. In certain embodiments such ablation of gene function can be utilized as part of a therapeutic strategy. In such cases the dominant-negative elements define protein therapeutic compositions. This is particularly applicable in those cases where the target proteins, from which the dominant-negative elements are derived, represent cell surface proteins. Further, a minimal dominant-negative element, in general, provides a smaller antigen than a larger protein, and the carrier protein of the dominant protein fragment can, for example, be chosen from a native, e.g., human, protein, thereby further reducing the likelihood of unwanted immunological problems.

### 5.2. The Target Protein

Generally, any target protein of interest may be employed to identify its specific protein-protein interactions, e.g., interactions with a particular protein, in accordance with the methods of the invention. In one embodiment, the methods of the invention are used to characterize novel genes identified by DNA sequencing approaches, for example, in the course of the human genome project. In another embodiment, the methods of the invention are employed to identify the function of proteins of interest that have been selected based on their particular expression pattern or suspected association with a particular disease.

The TDNE approach can also be used whether or not specific knowledge about a defined target protein exists. For example, differential expression analysis (e.g., Wang and Feuerstein, 1997, Cardiovasc. Res. 35:414-42; Winkles, 1998, Prog. Nucleic Acid Res. Mol. Biol. 58:41-78) can be used to determine which proteins are over expressed in a pathological state (such as a transformed cancerous state or an inflamed tissue). Differentially expressed proteins can then be analyzed for homodimeric or heterodimeric interactions. A homotypic interaction refers to an interaction in which the polypeptide partner is the same as the interacting portion of the protein-protein interaction domain. A heterotypic interaction refers to an interaction in which the polypeptide partner differs from the interacting portion of the protein-protein interaction domain.

For proteins that demonstrate interaction, the TDNE approach can be applied to target protein fragments that show (alone or as a member of a chimera) an interaction-blocking phenotype. Such identified fragment members can be expressed in a model or native system to evaluate the effect of the dominant-negative candidate on the pathological state. Any such TDNE that reverses (or partially reverses) the phenotype associated with the pathological state becomes a clear candidate for therapeutic intervention. Further, small molecules that mimic the TDNE dominant-negative activity can be identified via methods described herein and also represent candidates for therapeutic intervention in the case of the pathological situation in which the TDNE demonstrates a biological effect.

In another embodiment, for example, in instances in which a target protein is known to be involved in particular protein-protein interactions, the methods of the invention can be used to identify specific target protein regions that are involved in the protein-protein interactions of target protein. Among the target proteins are ones that participate in a protein-protein interaction that correlate with a particular pathological or are involved in a physiological process, required for its function. In various embodiments of the invention, the target protein can comprise an intracellular, extracellular, or a transmembrane protein, and/or a viral or other pathogenic protein. The target protein-protein interaction can be a homotypic or a heterotypic interaction.

In one embodiment, the target protein comprises a cell surface (transmembrane or membrane-associated) receptor. In instances in which interaction between the protein-protein interaction domain and its partner or partners is to be ligand-dependent, the ligand may or may not be introduced. In a specific embodiment, the assay systems described herein can be used to identify homotypic or heterotypic interactions, and to identify dominant-negative elements that modulate such interactions. Analysis in microbial assay systems can be used to determine whether homotypic or heterotypic dimerization takes place. Once a protein-protein interaction is demonstrated, the TDNE strategy described above can be used to identify tagged polypeptide fragments that block either the interaction. Specific examples include, e.g., leptin receptor co-expressed protein (LRCEP; Bailleul et al., 1997, Nucleic Acids Res. 25:2752-2758) and the leptin receptor. Other specific examples of cell surface receptors include, but are not limited to, members of the single transmembrane tyrosine receptor kinase (TRK)-like class of receptors (Ullrich & Schlessinger, 1990, Cell 61:203-12; Hunter & Cooper, 1985, Ann. Rev. Biochem. 54:897-930). In another embodiment, the target protein can be a member of the 7-transmembrane class of receptors (e.g., the G-protein coupled class of receptor (GPCR) (see Huang et al., 1997, J. Recept. Signal Transduct. Res. 17:599-607). In another embodiment, the target protein can be an ion channel membrane protein. Association between subunits of an ion channel provides a mechanism for the modulation of channel function (Ludewig et al., 1998, Nature 383:340-343; Fahike et al., 1997, J. Gen. Physiol. 109(1):93-104; Unwin, 1989, Neuron 3:665-76). Also provided are the voltage-gated ion channel family of receptors, such as the K⁺ sensitive channels and the Ca²⁺ sensitive channels (see, Hille, B. in “Ionic Channels of Excitable Membranes,” 1992, Sinauer Associates, Sunderland, Mass.; Catterall, W. A., 1991, Science 253:1499-1500, which are incorporated herein by reference in their entirety, and references cited therein). In another embodiment, the target protein can comprise a member of the receptor protein-tyrosine phosphatase family, or R-PTPs. Dimerization of this class of proteins may inhibit their activity, in contrast to the activating role that dimerization plays for many other receptors (see, e.g., Weiss & Schlessinger, 1998, Cell 94:277-80). In another embodiment, the target protein can comprise a member of the cytokine receptor family (see, e.g., Vigers et al. 1997, Nature 386:190-194). In another embodiment, the target protein can comprise a member of the nuclear hormone receptor superfamily (see, e.g., Mangelsdorf et al., 1995, Cell 83:835-39).

In various other embodiments, a cellular target protein may be chosen, such as protein involved in a signal transduction pathway. The methods described herein can be used to identify protein interaction domains, and molecules (e.g., chimeric dominant-negative peptides) that interrupt such interactions, that are important for various steps within signal transduction pathways from the cell surface receptor to transcriptional activation in the nucleus. Many such cellular proteins are known to have domains and motifs, such as SH2, SH3, PH, PTB, WW, and WD40 domains, leucine-rich repeats, and F-box motifs, that are involved in cellular protein-protein interactions (see Sudol et al., 1996, Trends Biochem. 21:1-3, and Koch et al, 1991, Science 252:668-74). In a specfic embodiment, the target protein can comprise a heterotrimeric G-protein (Neer, 1995, Cell 80:249-257; Clapham & Neer, 1993, Nature 365:403-406). In another embodiment, the target protein can comprise a non-receptor protein kinases, such as the src family or the Janus family of protein tyrosine kinases (see, e.g., Darnell et al., 1994, Science, 264:1415-21,; Cantley et al., 1991, Cell 64:281-302). Further, in another embodiment, the target protein can comprise a transcription factor protein. Many transcription factors are activated by homotypic and heterotypic dimerization (see, e.g., Lamb & McKnight, 1991, Trends Biochem. Sci. 16:417-22 ). Thus, target proteins can include, for example, transcription factors containing leucine zipper dimerization domains, including, but not limited to Fos/Jun (Bohmann et al., Science 238:1386-92; and Angel et al., 1988, Nature 332:166-71), C/EBP (Landshultz et al., 1988, Science, 240:1759-64), GCN4 (see, e.g., Agre et al., 1989, Science 246:922-926); helix loop helix (HLH) domain proteins, for example Myc (Murre et al, 1989, Cell 56:777-783) and MyoD and other myogenic HLH proteins which require heterooligimerization with E12/E47-like proteins in vivo (Lasser et al., 1991, Cell 66:305-15), as well as other dimerizing transcription factors well known in the art.

The methods of the invention can also be used to identify and interupt protein-protein interactions important in intercellular interactions. For example, cell adhesion proteins such as integrins, may require association with partner disintegrin proteins (Blobel, 1997, Cell 90:589-92). In this specific embodiment, the target protein integrin protein can be used to identify a disintigrin partner, or an inhibitory molecule of an integrin-disintegrin interaction.

In yet another embodiment, the target protein can comprise a protein derived from a virus, microbe, or other pathogen. For example, peptide inhibitors of dimerization of HIV protease can be identified using the methods of the invention with HIV protease as a target protein (McKeever et al., 1989, J. Biol. Chem. 264:1919-1921).

In addition to the proteins mentioned herein, a target protein can comprise amino acid residues derived from any dimeric or multimeric polypeptide listed in public databases, such as, for example, the Swiss Protein Data Base (SWISS-PROT; see Bairoch & Apweiler, 1998, Nucl. Acids Res. 26:38-42; see also http://www.expasy.ch and rhttp:/www.ncbi.nlm.nih.gov).

### 5.3. Tag Dominant-Negative Elements (TDNEs), TDNE Libraries, and Generation Thereof

The present invention provides for TDNEs, expression vectors encoidng TDNEs and libraries comprising a multipliciy of candidate TDNEs or TDNE expression vectors, and methods for constructing the same.

A TDNE comprises a candidate protein fragment or peptide, e.g., a fragment derived from a target protein of interest, such a a target protein involved in a particular protein-protein interaction of interest, fused to a carrier protein, or “tag”. The TDNE tag can be present either amino to or carboxy to the protein fragment, e.g., the target protein fragment. The TDNE tag and protein fragment portions are fused or operably attached via standard linkages, e.g., via peptide bond linkages. Particular linkages appropriate for the specific tag elements described below are well known to those of skill in the art.

A TDNE library can comprise a plurality of TDNE fusion proteins, with at least a subset of the TDNE library members comprising protein fragment portions that differ one from the other. In one embodiment, a TDNE library comprises a plurality of TDNE fusion proteins wherein the protein fragment portions of the TDNE library members are derived from a particular target protein of interest. Preferably, in such an embodiment, the protein fragment portions, in toto, represent all or substantially all of the amino acid sequence of the target protein of interest, or all or substantially all of the portion of the target protein known of suspected of being involved in the protein-protein interaction of interest. A TDNE library can also comprise a plurality of TDNE expression vectors, each of which express a candidate protein fragment or peptide derived from a target protein of interest, fused to a carrier protein, or “tag”. A TDNE library can still further comprise a plurality of cells, e.g., microbial cells, at least a subset of which contain different TDNE fusion protein members and/or contain different TDNE expression vectors that are expressed or can be expressed in the cells.

Although the protein fragment can be of any length, e.g., can approach the full-length size of the target protein (e.g., about 80% or more of the entire target protein of interest), this portion of the TDNE typically has a length of about six (6) to about 500 amino acids, preferably it has a length of about six (6) to about fifty (50) amino acids or of about six (6) to about 150 amino acids, and most preferably it has a length of about six (6) to about thirty (30) amino acids. The optimal length of the protein fragment will vary depending on the specific structure of the protein to be examined and can routinely be determined in that the microbe-based strategies and systems described herein allow for rapid empirical determination of the preferred size.

A variety of peptide tags known in the art may be used as tags in TDNE fusion proteins, including but not limited to the polyhistidine sequence (Petty, 1996, Metal-chelate affinity chromatography, in Current Protocols in Molecular Biology, Vol. 2, Ed. Ausubel et al., Greene Publish. Assoc. & Wiley Interscience), glutathione S-transferase (GST; Smith, 1993, Methods Mol. Cell Bio. 4:220-229), the E. coli maltose binding protein (Guan et al., 1987, Gene 67:21-30), and various cellulose binding domains (U.S. Pat. Nos. 5,496,934; 5,202,247; 5,137,819; Tomme et al., 1994, Protein Eng. 7:117-123), etc. Other possible peptide tags are short amino acid sequences to which monoclonal antibodies are available, such as but not limited to the following well known examples: the FLAG epitope, the myc epitope at amino acids 408-439, the influenza virus hemaglutinin (HA) epitope. Other peptide tags are recognized by specific binding partners and thus facilitate isolation by affinity binding to the binding partner, which is preferably immobilized and/or on a solid phase surface. As will be appreciated by those skilled in the art, many methods can be used to obtain the coding region of the above-mentioned peptide tags, including but not limited to, DNA cloning, DNA amplification, and synthetic methods. Some of the peptide tags and reagents for their detection and isolation are available commercially. DNA sequences encoding desired peptide tags which are known or readily available from libraries or commercial suppliers are suitable in the practice of this invention.

Peptide tags designed to target proteins to the inner cell membrane can also be used. Leader sequences, associated with proteins naturally destined for the periplasm, are, for example, known to direct the secretion of foreign proteins to the periplasm (MacIntyre et al., 1990, Mol. Gen. Genet. 221:466-474). Such tags are particularly important for use in connection with the CadC-based assays described in Section 5.4.1, below, to deliver candidate dominant-negative elements to the periplasmic space. In a preferred embodiment, the tag comprises the OmpA protein leader sequence (Hobom et al., 1995, Dev. Biol. Stand. 84:255-262). Other signal leader sequences are also possible, including, but not limited to, the leaders from E. coli PhoA (Oka et al., 1985, Proc. Natl. Acad. Sci 82:7212-16), OmpT (Johnson et al., 1996, Protein Expression 7:104-113), LamB and OmpF (Hoffman & Wright, 1985, Proc. Natl. Acad. Sci. USA 82:5107-5111), β-lactamase (Kadonaga et al., 1984, J. Biol. Chem. 259:2149-54), enterotoxins (Morioka-Fujimoto et al., 1991, J. Biol. Chem. 266:1728-32), protein A from Staphylococcus aureus (Abrahmsen et al., 1986, Nucleic Acids Res. 14:7487-7500), endoglucanase from B. subtilis (Lo et al., Appl. Environ. Microbiol. 54:2287-2292), as well as artificial and synthetic signal sequences (MacIntyre et al., 1990, Mol. Gen. Genet. 221:466-74; Kaiser et al., 1987, Science, 235:312-317).

In one embodiment, for example, a candidate TDNE can be used to characterize the gene product from which the dominant-negative element was derived. In this case, the tag may comprise a flourescent peptide, such as GFP (green fluorescent protein), that can be used to identify the target gene product in a particular sub-cellular compartment, for example, by fluorescence microscopy (Flach et al., 1994, Mol. Cell. Biol. 14:8399-8407). In another embodiment, a tag comprising an antigenic peptide could be used to allow immunohistochemical staining of cells, using techniques which are well known in the art (see Antibodies: A Laboratory Manual, Harlow and Lane (eds.), Cold Spring Harbor Press, Cold Spring Harbor, N.Y.,1988). Such antigenic peptide tags can also be used to confirm that a candidate TDNE interacts with the target protein in vivo. In this embodiment, the TDNE could be expressed in appropriate host cells containing the target protein, and coimmunoprecipitation experiments can be used to identify in vivo and in vitro interactions (Phizicky and Fields, 1995 Microbiol. Rev. 59:94-123). Other tags can be employed that aid in the determination of the three dimensional structure of the TDNEs through standard structure determination methods like x-ray crystallography or nuclear magnetic resonance (NMR). Structural determination of the TDNEs that are promising therapeutic candidates further aid in the development of small molecules that mimic the affect of the TDNEs through structure based drug design approaches.

TDNE fusion proteins are encoded by and can be expression via TDNE expression vectors which are intended to be part of the present invention. A TDNE expression vector comprises an origin of replication suitable for the contemplated host cell (e.g., bacterial or yeast host cell), a selectable marker, and nucleotides sequences required for expression, preferably inducible expression, of a candidate TDNE within the contemplated host cell.

Details for representative, non-limiting teaching for the construction of TDNE expression vectors and libraries are described hereinbelow.

First, appropriately sized DNA fragments derived from nucleic acid sequences encoding the target protein of interest are prepared. Fragments may be derived from a nucleic acid encoding the target protein of interest, or from another source. Typically, DNA fragments are chosen such that their encoded polypeptides have a length of about six (6) to about 500 amino acids, preferably it has a length of about six (6) to about fifty (50) amino acids or about six (6) to about 150 amino acids, and most preferably it has a length of about six (6) to about thirty (30) amino acids. α-donor discussed above, the optimal length of the protein fragment acting as dominant-negative element will vary depending on the specific structure of the protein to be examined, but the disclosed microbe-based strategies allow for rapid empirical determination of the preferred size, which may vary form six to twelve amino acids, however, may be as large as a size approaching that of the full-length protein of interest (e.g., greater than about 80% of the full-length size).

Methods for preparing appropriately sized DNA fragments include, but are not limited to: the ultrasonic disruption of a DNA fragment encoding a target protein into fragments, preferably fragements in the size range of 200-700 base pairs, the use of randomly primed PCR products from the target gene of interest, and the construction of families of fragments using an set of restriction nuclease treatments employing nucleases that show an array of four base pair sequence specificities.

In a specific embodiment, TDNE expression vectors and libraries can be constructed using the target gene fragmented by partial digestion with the restriction nuclease CviJI under “relaxed” conditions where it acts to cleave at PyGCPy, PuGCPu and PuGCPy sites (Fitzgerald et al., 1992, Nucleic Acids Res. 20:3753-3762). Alternatively, near-random cleavage could also be produced using a mixture of other restriction endonucleases. Partial digestion to produce judiciously sized pieces (40-500 nucleotides for the 12-150 amino acid protein fragments) can be assayed and/or size-selected using gel electrophoresis. Collections of such pieces can be cloned in an appropriate vector to produce the required fragment libraries (either alone or as fusion proteins).

The second step in the construction of the expression vectors and libraries of TDNEs involves selection of a “tag” polypeptide to be used in construction of the fusion protein. The choice of the tag polypeptides will depend on the desired application. The tags may be selected to accomplish various functions including, but not limited to, subcellular localization of TDNEs, facilitation of recovery and/or purification of TDNEs, cross-linking of TDNEs to neighboring proteins, and immunoprecipitation of protein complexes. Alternatively, the tag may simply serve as carrier protein to add stability to the candidate dominant-negative element, or provide an additional steric block to a protein interaction. Non-limiting examples of possible tag elements are described above.

In one aspect of the invention, the TDNE expression vector can be designed for modularization of functional tags. Various TDNE expression vectors with compatible cloning sites can be constructed comprising sequences encoding tags designed for different purposes. Sequences encoding a candidate dominant negative element can then be shuttled from one vector to another to express a candidate element fused to different functional tags. For example, a candidate TDNE can be shuttled from a first vector encoding a tag used for stabilization or cellular localization in a primary screening assay, to a second vector encoding an antigenic tag that can be used for immunprecipation or immunohistochemical localization of TDNE-protein complexes. Methods and compositions appropriate for routine shuttling of sequences between vectors are well known to those of skill in the art.

Next, the DNA fragments derived from genes encoding the target protein of interest, or other candidate dominant-negative element, and the nucleotide sequences encoding tags are cloned into an appropriate expression vector, either together or in separate cloning steps using standard molecular biology techniques (see e.g., Methods in Enzymology, 1987, volume 154, Academic Press; Sambrook et al. 1989, Molecular Cloning—A Laboratory Manual, 2nd Edition, Cold Spring Harbor Press, New York; and Current Protocols in Molecular Biology, Ausubel et al. (eds.), Greene Publishing Associates and Wiley Interscience, New York, each of which is incorporated herein by reference in its entirety).

As discussed above, a TDNE expression vector comprises an origin of replication, a selectable marker, and suitable regulatory elements and cloning sites for insertion and expression of nucleotide sequences encoding the tag and candidate dominant negative element, such that insertion of such sequences results in the ability to express the TDNE, preferably in a regulated fashion, in a host cell of interest (e.g., a microbial cell such as a bacterial or yeast cell).

For cloning and propagation in E. coli, any E. coli origin of replication may be used, examples of which are well-known in the art Non-limiting examples of readily available plasmid origins of replication are ColE1-derived origins of replication (Bolivar et al., 1977, Gene 2:95-113; see Sambrook et al., 1989, supra), p15A origins present on plasmids such as pACYC184 (Chang & Cohen, 1978, J. Bacteriol. 134:1141-56; see also Miller, 1992, A Short Course in Bacterial Genetics, Cold Spring Harbor Laboratory Press, NY p10.4-10.11), and pSC101 origin available for low-copy plasmids expression are all well known in the art. One example of a high-copy number plasmid comprises an origin from pUC19 and its derivatives (Yanisch-Perron et al., 1985, Gene 33:103-119). pUC vectors exist at levels of 300-500 copies per cell and have convenient cloning sites for insertion of foreign genes.

For expression of TDNE fusion proteins, the TDNE expression vector further comprises DNA control elements which control expression, preferably regulated or inducible expression, over a range of different expression levels. A variety of such regulatory sequences are well known to those of skill in the art. The ability to generate a wide range of expression is advantageous for utilizing the methods of the invention, as described below.

Inducible expression yielding a wide range of expression can be obtained by utilizing a variety of inducible regulatory sequences. Levels of expression from TDNE library expression constructs can also be varied by using promoters of different strengths. In one embodiment, for example, the lacI gene and its gratuitous inducer IPTG can be utilized to yield inducible, high levels of expression of TDNE libraries when sequences encoding such polypeptides are transcribed via the lacOP regulatory sequences. Other regulated expression systems that can be utilized include but are not limited to, the araC promoter which is inducible by arabinose (AraC), the TET system (Geissendorfer and Hillen, 1990, Appl. Microbiol. Biotechnol. 33:657-663), the p_{L} promoter of phage λ temperature and the inducible lambda repressor CI₈₅₇ (Pirrotta, 1975, Nature 254: 114-117; Petrenko et al, 1989, Gene 78:85-91), the trp promoter and trp repressor system (Bennett et al., 1976, Proc. Natl. Acad. Sci USA 73:2351-55; Wame et al., 1986, Gene 46:103-112), the lacUV5 promoter (Gilbert and Maxam, 1973, Proc. Natl. Acad. Sci. USA 70:1559-63), lpp (Nokamura et al., et al., 1982, J. Mol. Appl. Gen. 1:289-299), the T7 gene-10 promoter, phoA (alkaline phosphatase), recA (Horii et al. 1980), and the tac promoter, a trp-lac fusion promoter, which is inducible by tryptophan (Amann et al., 1983, Gene 25:167-78), for example, are all commonly used strong promoters, resulting in an accumulated level of about 1 to 10% of total cellular protein for a protein whose level is controlled by each promoter. If a stronger promoter is desired, the tac promoter is approximately tenfold stronger than lacUV5, but will result in high baseline levels of expression, and should be used only when overexpression is required. If a weaker promoter is required, other bacterial promoters are well known in the art, for example, maltose, galactose, or other desirable promoter (sequences of such promoters are available from Genbank (Burks et al. 1991, Nucl. Acids Res. 19:2227-2230).

Induction of TDNE expression is, preferably, inducible in a manner independent of expression of the target gene polypeptide (e.g., expression of one construct is lac-inducible and the other is ara-inducible). In addition, the chosen vector must be compatible with the constructs used in protein-protein interaction assays described in Section 5.4, below. One of skill in the art would readily be aware of the compatibility requirements necessary for maintaining multiple plasmids in a single cell. Methods for propagation of two or more constructs in microbial cells are well known to those of skill in the art. For example, cells containing multiple replicons can routinely be selected for and maintained by utilizing vectors comprising appropriately compatible origins of replication and independent selection systems (see, Sambrook et al., 1989, supra, and references therein; and Miller, 1992, A Short Course in Bacterial Genetics, Cold Spring Harbor Laboratory Press, NY).

With respect to yeast expression and vectors utilized for expression in yeast, appropriate origins of replication, i.e., 2 μm circle-derived vectors, regulatory sequences for expression, both constitutive and regulatory, e.g., inducible, expression. Starting cells for producing yeast cell-based systems of the invention can be obtained, e.g., from the sources listed earlier in this Section, above. Standard techniques can be utilized for manipulation and maintaining yeast cells. Standard techniques can also be utilized for recombinant expression, including regulatable expression, can also be utilized. See, e.g., Kaiser, C., 1994, “Methods in Yeast Genetics,” Cold Spring Harbor Laboratory Press, New York; and Spenser, J. F. T., 1989, “Yeast Genetics,” Springer-Verlag, New York, each of which is incorporated herein by reference in its entirety.

### 5.4. Assay Systems for the Identification of Tag Dominant-Negative Elements

A number of assay systems can be employed for the identification of dominant-negative elements which bind and/or exhibit inhibitory activity on the target protein of interest. Typically, an assay system comprises a cell containing a TDNE fusion protein and/or a TDNE expression vector that is or can be expressed in the cell, preferably in a regulatable, e.g., inducible, fashion. The system typically further comprises a partner protein involved in the protein-protein interaction of interest, or the portion of the partner protein that participates in the protein-protein interaction, and/or an expression vector that expresses or can express the partner protein or portion thereof in the cell. The assay system further comprises elements, as described below, that allow for an assay of the protein-protein interaction of interest.

Typically, such assay systems respond to a protein-protein interaction or its disruption by a detectable signal. The detectable signal may be expression of an identifiable gene product, which is activated in response to the protein-protein interaction or its disruption. For example, the protein of interest (X) and a pool of dominant-negative element candidates (Yₓ, composed of Y₁, Y₂, . . . , Yₙ) may be expressed in a suitable cellular system, wherein each component is fused to a DNA-binding domain (DB) or an activation domain (AD), respectively, such that molecules of the structure DB-X and AD-Yₓ are formed within the cell. If DB-X finds an AD-Y binding partner, the resulting DB-X/AD-Y interaction will reconstitute a functional transcription factor that activates a reporter gene driven by a promoter containing DB binding sites.

In an alternative embodiment, the target protein of interest, X, may be fused to an activation domain, AD, such that AD-X is formed, and co-expressed with TDNE library, DB-Yₓ, composed of potential dominant-negative elements, Yₓ, fused to a DNA-binding domain, DB. TDNEs which bind to the target protein of interest, X, may be identified by activation of the reporter gene system.

In another embodiment, each of two target proteins of interest which are known to interact in vivo, X and Z, may be fused to one of DB and AD, respectively, such that DB-X and AD-Z is formed. Co-expressing DB-X and AD-Y results in constitutive activation of the reporter gene system. Co-expression of members of a [T]DNE library (using such approach, the dominant-negative elements do not necessarily have to be fusion proteins, although fusion proteins are preferred, see, supra) which interrupt such interaction accordingly will result in suppression of expression of the reporter gene system. [T]DNEs which inhibit interaction of X and Z may thus be identified.

A number of systems have been described which can be adapted for the identification of dominant-negative elements. One well known system is the yeast two-hybrid system (Fields and Song, 1989, Nature 340:245-246; White. 1996, Proc. Natl. Acad. Sci. USA 93:10001-10003; Warbick, 1997, Structure 5:13-17) which has been used to identify interacting proteins and to isolate the corresponding encoding genes. In this system, prototrophic selectable markers which allow positive growth selection are used as reporter genes to facilitate identification of protein-protein interactions. Applying the above general scheme, growing yeast cell colonies expressing DB-X/AD-Y-interacting proteins can be identified among the non-growing colonies (Gyris et al., 1993, Cell 75:791-803; Durfee et al., 1993, Genes Dev. 7:555-569; Vojtek et al., 1993, Cell 74:205-214). Related systems which may be employed include the yeast three-hybrid system (Licitra and Liu, 1996, Proc. Natl. Acad. Sci. USA 93:12817-12821; Tirode et al., 1997, J. Biol. Chem. 272:22995-22999) and the yeast reverse two-hybrid system (Vidal et al., 1996, Proc. Natl. Acad. Sci. USA 93:10321-10326; Vidal et al., 1996, Proc. Natl. Acad. Sci. USA 93:10315-10320).

Bacterial systems for identification of protein-protein interactions are also known in the art and may be adapted for use with the methods of the present invention. For example, in one embodiment, as described in detail below, the E. coli CadC-based dimer detection system may be used for identifying dominant-negative elements (PCT publication no. WO 99/23116 dated May 14, 1999, which is incorporated herein in its entirety). In another embodiment, a bacterial protein interaction system based on the AraC protein, which regulates the L-arabinose operon in E. coli, may be used (Bustos and Schleif, 1993, Proc. Natl. Acad. Sci. USA 90:5638-5642; Soisson et al., 1997, Science 276:421-425; Eustance et al., 1994, J. Mol. Biol. 242:330-338). Other assay systems which may be used include bacterial systems based on the lambda repressor system (Zeng et al., 1997, Protein Sci. 6:2218-2226), the lac-operon (Gates et al., 1996, J. Mol. Biol. 255:373-386), an interaction signal detection based on lambda and lambdoid proteins (Hollis et al., 1988. Proc. Natl. Acad. Sci. USA 85:5834-5838), systems based on E. coli RNAP (Dove et al., 1998, Genes Dev. 12:745-754; Dove et al., 1997, Nature 386:627-630), and systems based on the cAMP synthetase (Karimova et al., 1998, Proc. Natl. Acad. Sci. USA 95:5752-5756). Systems based on other bacterial proteins, may be developed to detect and quantify protein-protein interactions. Such systems could be modified to allow the co-expression of target protein fragment families fused to carrier protein which would allow the detection of TDNE that block the protein-protein interaction of interest.

The general principles of assays suitable for the identification of TDNEs that interfere with binding of a target protein are described in detail below. In particular, this section describes the use of the E. coli-based CadC dimerization detection system, the AraC-based homodimer detection system, and the yeast two-hybrid system.

5.4.1. The E. coli CadC Dimerization Detection Assays And Their Use In Identifying Dominant-Negative Elements

CadC is a dual functional single transmembrane receptor protein found in E. coli and other phylogenetically related species. In E. coli, the function of CadC is to sense environmental signals of pH and lysine and respond by modulating transcription from the cadBA operon (Meng et al., 1993, J. Bacteriol. 175:1221-1234). CadC is composed of three functional domains: a periplasmic sensing domain (PSD), a transmembrane domain (TMD) and a cytoplasmic transcriptional regulator domain (TRD). Transcriptional activation of the cadBA regulatory region requires interaction, i.e., dimerization, between CadC periplasmic domains. Thus, expression levels of a reporter gene, operatively linked to the cadBA regulatory region, can be measured to detect the level of CadC dimerization.

This system has been used to develop a dimerization-dependent expression system that can be used to assay protein-protein interactions in any target protein of interest (PCT publication no. WO 99/23116). CadC-fusion polypeptides are constructed by linking sequences encoding the CadC activation and transmembrane domains to sequences encoding a candidate protein-protein interaction domain derived from a known target gene of interest, or an unknown test gene. Dimerization, either homotypic or heterotypic, of the periplasmic domain, results in transcriptional activation of the cadBA regulatory region and expression of a reporter gene sequence. A CadC-responsive “reporter gene” sequence can comprise any gene sequence which expresses or encodes a detectable gene product (RNA or protein). Such a gene product is detectable either by its presence, or by its activity that results in the generation of a detectable signal. A reporter gene is used in the invention to monitor the ability of a test compound to activate the CadC transcriptional regulatory domain. For example, in a preferred embodiment, enzymatic reporters and light-emitting reporters analyzed by colorometric or fluorometric assays are preferred for the screening assays of the invention. Such reporter genes include, but are not limited to β-galactosidase (Nolan et al. 1988, Proc. Natl. Acad. Sci. USA 85:2603-07), β-glucuronidase (Roberts et al. 1989, Curr. Genet. 15:177-180), luciferase (Miyamoto et al., 1987, J. Bacteriol. 169:247-253), or β-lactamase. In one embodiment, the reporter gene sequence comprises a nucleotide sequence which encodes a LacZ gene product, β-galactosidase. The enzyme is very stable and has a broad specificity so as to allow the use of different histochemical, chromogenic or fluorogenic substrates, such as, but not limited to, 5-bromo-4-chloro-3-indoyl-β-D-galactoside (X-gal), chlorophenol red β-D-galactoside (CPRG, Eustice, et al, 1991, Biotechniques 11: 739-742), lactose 2,3,5-triphenyl-2H-tetrazolium (lactose-tetrazolium), and fluorescein galactopyranoside (see Nolan et al., 1988, supra). The Examples presented in Sections 6.4 and 6.5 demonstrate the successful identification of a TDNE-mediated interuption of a CadC-fusion polypeptide interaction by measuring the levels of a β-galactosidase reporter gene activity. A variety of other reporter gene sequences well known to those of skill in the art can be utilized, including bioluminescent, chemiluminescent and fluorescent proteins, or proteins that confer antibiotic resistance such as chloramphenicol transacetylase (CAT) can be utilized. Other selectable reporter gene sequences can also be utilized and include, but are not limited to gene sequences encoding polypeptides which confer zeocin (Hegedus et al. 1998, Gene 207:241-249) or kanamycin resistance (Friedrich & Soriano, 1991, Genes. Dev. 5:1513-1523). CadC-fusion polypeptides and cadBA reporter constructs can be constructed according to standard recombinant DNA techniques (see e.g., Methods in Enzymology, 1987, volume 154, Academic Press; Sambrook et al. 1989, Molecular Cloning—A Laboratory Manual, 2nd Edition, Cold Spring Harbor Press, New York; and Ausubel et al. Current Protocols in Molecular Biology, Greene Publishing Associates and Wiley Interscience, New York).

For use in a TDNE screening assay, a CadC fusion polypeptide is constructed using a gene sequence encoding a target protein of interest for the CadC fusion polypeptide periplasmic domain. Optionally, where a heterotypic interaction is being analyzed, a second CadC fusion polypeptide is constructed, using a partner of the target protein for the CadC fusion polypeptide periplasmic domain. In addition, a TDNE library is constructed, comprising a plurality of TDNE vectors that are capable of expressing fragments of the target protein, or alternatively, fragments of the partner of the target protein. The CadC fusion polypeptides are then expressed in a cell containing both the CadC fusion polypeptides and the CadBA reporter gene construct. Once conditions are found such that a positive signal is detected from the reporter gene, the TDNE library is induced for expression and cells are plated on selective media. In instances wherein co-expression results in a relative decrease in reporter gene expression, a candidate TDNE is identified. Cells in which the positive signal has been diminished are then chosen, and the candidate TDNE plasmid DNA is isolated and further analyzed.

5.4.2. Yeast-Based Systems To Identify Dominant-Negative Elements

While the above described CadC-based system provides a means to rapidly test and define interactions that will lead to the identification of dominant-negative elements for many classes of proteins, there are certain limitations to expression and analysis of eukaryotic proteins in bacterial cells. For example, in cases where post-translational modifications, such as, but not limited to phosphorylation, glycosylation, palnitoylation, myristilation, or proteolytic processing events are important for the formation and stability of protein-protein interactions, a eukaryotic host is preferable. According to the present invention, TDNE-related modifications of yeast-based systems have been developed suited to determine and characterize both heterotypic and homotypic protein-protein interactions.

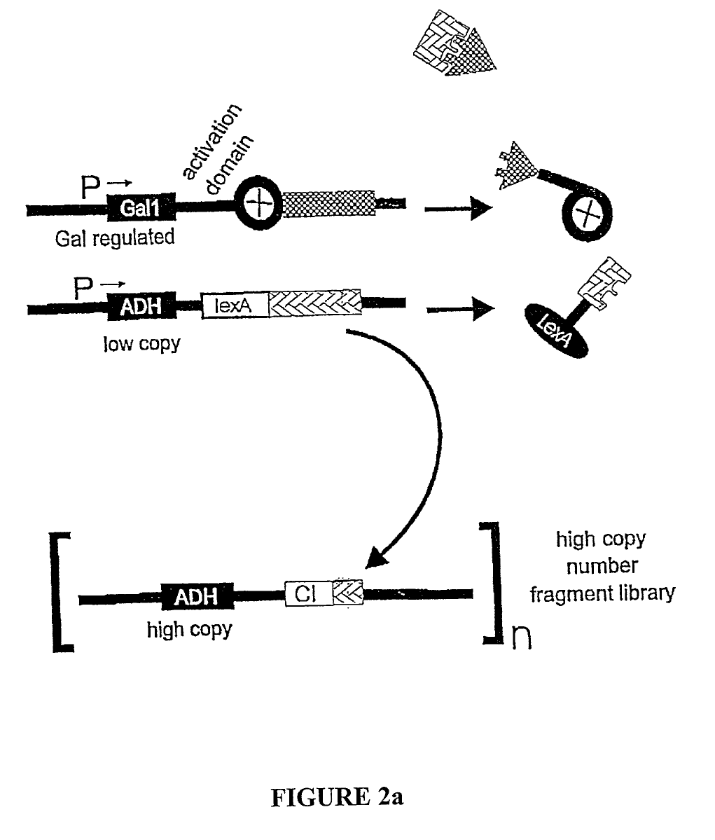

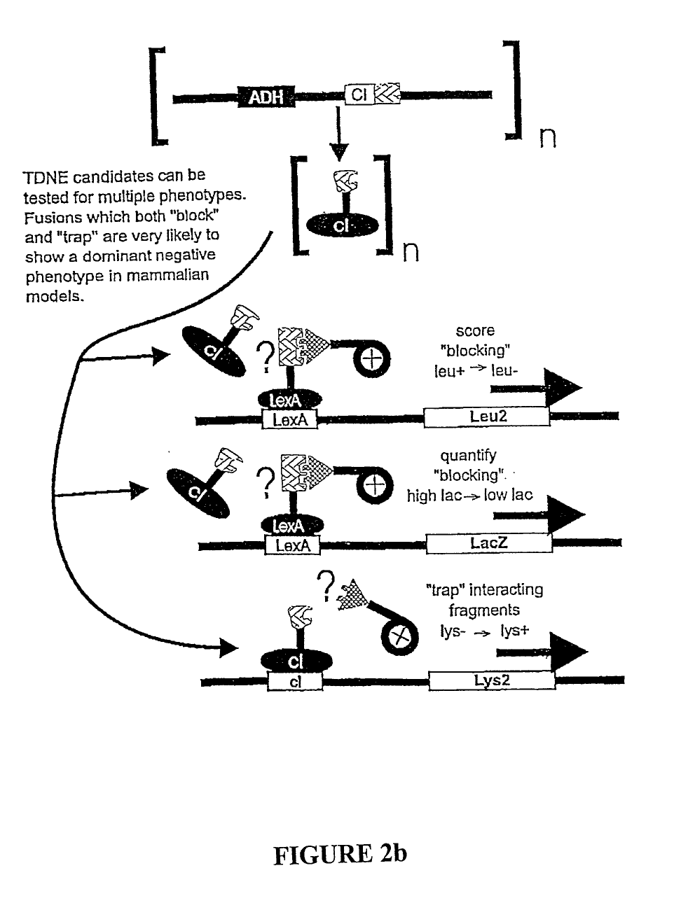

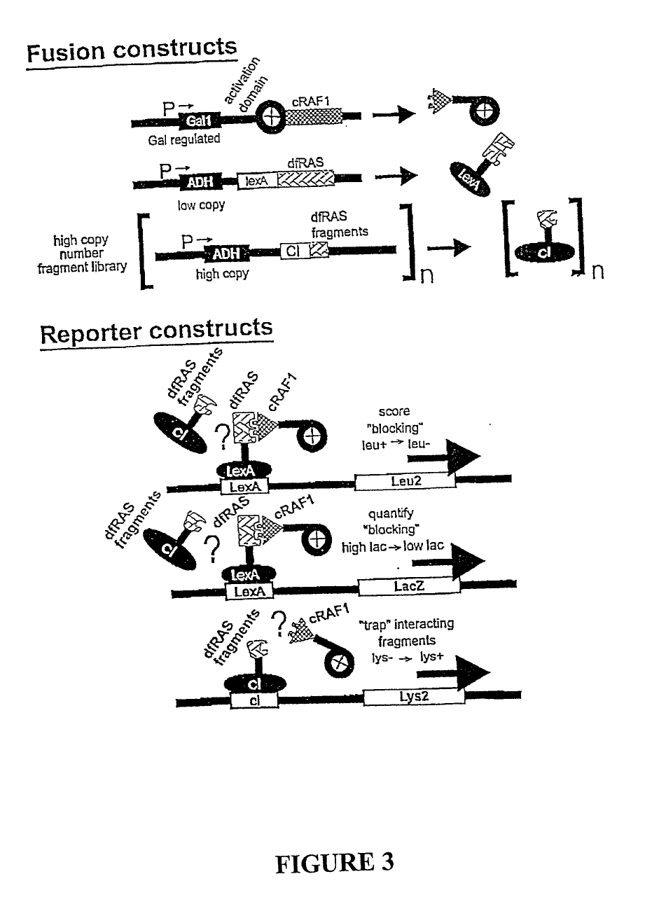

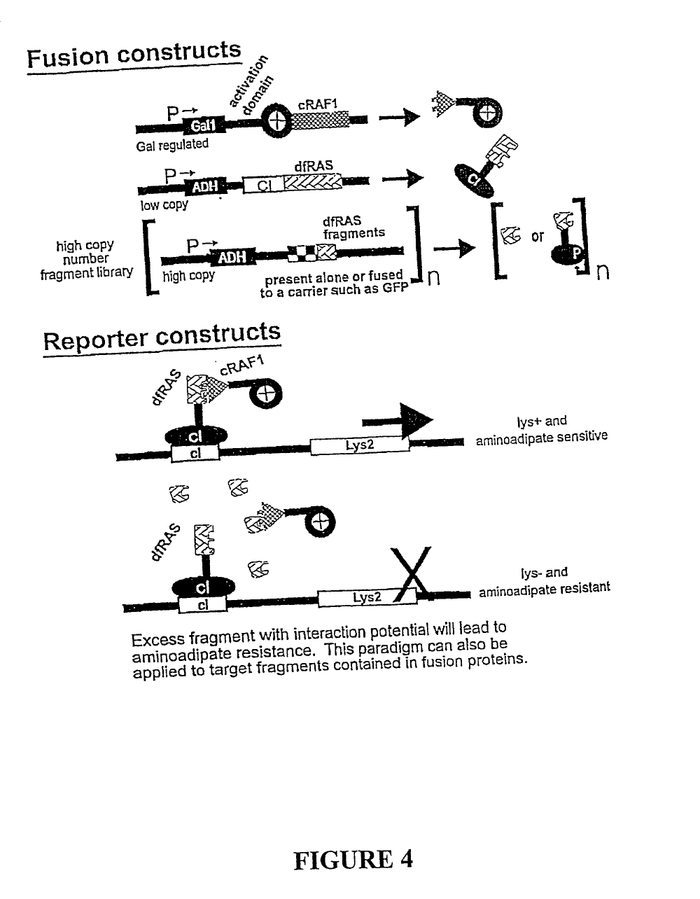

In one embodiment, a TDNE-related modification of a yeast dual-bait system can be employed and adapted to screen for dominant-negative elements which bind to and/or inhibit a target protein of interest. This system, as modified and adapted for the methods of the present invention, is depicted in FIG. 2.

For a non-limiting approach according to the present invention, such a system can comprise the following:

(1) a first reporter gene linked to a LexA binding operator site;

(2) a second reporter gene linked to the same binding site provided in (1);

(3) a third reporter gene linked to a cI binding operator site;

(4) a first fusion gene and consisting of LexA and a partner protein of interest (or a portion thereof involved in the protein-protein interaction of interest) for which an interaction has been previously established;

(5) a second fusion gene that encodes a protein containing a transcriptional activation domain protein fused to the target protein (or a portion thereof involved in the protein-protein interaction of interest) of the partner protein of (4);

(6) a TDNE or library of TDNEs derived from either the partner sequence of (4) or the target sequence of (5) fused to cI.

Any reporter gene sequence can be utilized herein and/or listed in FIG. 2, can be utilized for the above system. The activities of the products of the reporter gene sequences of (1), (2) and (3) must be distinguishable from each other.

In this system, in order for the reporter genes of (1) and (2) to be expressed, the protein expressed by the sequence of (4) and the protein expressed by the sequence of (5) must interact such that the resulting complex binds to and activates expression of (1) and (2). The interaction of the proteins of (4) and (5) occurs via the protein-protein interaction of interest.

A candidate TDNE that modulates the protein-protein interaction of interest can be identified, therefore, by co-expressing the sequences of (4), (5) and (6) in the above system and assaying for reporter gene expression relative to levels obtained in the system in the absence expression of the sequences of (6). See FIG. 2B. In instances wherein co-expression results in a relative decrease in (1) and (2) reporter gene expression, a candidate TDNE is identified. Likewise, an increase in (3) reporter gene expression also identifies a candidate TDNE.

The observation of such a “trap” phenotype for the cI fusion protein (3), by definition, assures an in-frame fusion, and in some instances the reporter gene will allow the direct selection of an in-frame fusion with a “trap” phenotype. In this configuration, the strain in which the “trap” interaction has been isolated is also scored for a “blocking” phenotype, as observed via the decrease in (1) and (2) expression.

The above outlined approach, based on the system, therefore provides a microbe-based strategy to identify candidate TDNEs that will block protein-protein interactions of interest and thereby identify TDNEs that will act as dominant-negative sequences. The disruption of the native tertiary structure of a cognate parental protein by a dominant-negative element is expected to lead to the ablation of function. Ablation of function will allow for a test of a hypothesized function. Moreover, ablation of function may lead to the discovery of a protein's function even in the absence of a hypothesis. In addition to providing a chimera that can assess function in model systems, inhibitory TDNEs identified with this system may also have value as a therapeutic agent in those cases where the ablation of function has a desirable therapeutic endpoint.

Interacting TDNE chimera also have the potential to be used as cytological reagents by virtue of their demonstrated ability to interact with the domains of interest. The procedures employed to define the TDNE's interaction potential, i.e., its behavior in the “trap” and “blocking” assays, will guide the interpretation of any cytological observation. The common cIportion of all TDNEs derived from this strategy will allow a single antibody-based reagent to be used in all work. Information about the subcellular location and tissue distribution of a new protein will further help define its function.

When the identified TDNE interferes with a defined a function of either the partner protein, the same system as originally employed may be used to identify compounds, e.g., small molecules, that block the same protein-protein interaction that initially uncovered the inhibitory effect of the candidate TDNE. In this approach, two different interactions can be examined and two outcomes are possible for a compound that reverses the “trap” interaction. A compound could be competent to block interactions of the activator fusion with both the truncated TDNE domain, fused to cI, and the intact domain, i.e., the original LexA fusion. In this instance, the reporter response from the cI-linked reporter is lost and the response from the LexA-linked reporter remains inactive. In this scenario, the LexA-linked reporter was originally inactive because the cI fusion competed for the activator, and remains inactive because the compound which disrupted the cI interaction, with the truncated domain, also blocks the interaction the full-length LexA fusion. It is also possible that a compound can block the interaction with the truncated cI fusion but fails to block a more extensive interaction with the full-length LexA fusion. Such a compound lead sto the loss of cI reporter activity and reestablishment the LexA reporter signal.

The system described herein can be used not only to identify compounds, but also to characterize them. Different strategies would be used in the development of the compounds that fit the two categories described above. In both cases, however, the compound would clearly be of interest because it had demonstrated the capacity to block an interaction that was first shown to be critical by virtue its ability to provide the contacts necessary to form the basis of a dominant-negative fusion protein. In one specific non-limiting example of such a system of the invention, the system includes a specific yeast background comprising a yeast-based modified dual-bait scheme for the identification of TDNEs derived from human HA-Ras, human c-Raf1 and human activated Raf1.

The system for the identification of TDNEs from human HA-Ras comprises:

(1) a LexA-dfRas (df for mutagenized CAAX box) fusion gene integrated into the yeast SKY191 chromosome and placed under the control of the ADH promoter region;

(2) cI-operator-LacZ reporter fusion gene on a two (2) micron plasmid under the control of an activator dependent minimal Gal1 promoter region;

(3) Activation Domain-c-Raf1 fusion gene (AD-cRaf1) on a two (2) micron plasmid under the control of galactose inducible Gal1 promoter;

(4) LexA-operator-LacZ reporter fusion gene on a two (2) micron plasmid under the control of an activator dependent minimal Gal1 promoter region; and

(5) cI-TDNE fusion library on a two (2) micron multicopy plasmid under the control of the ADH promoter region.

When SKY-191 yeast cells with the integrated LexA-dfRas fusion gene and the activation domain-cRaf1 fusion gene on two (2) micron plasmid are grown on media with galactose, both fusion proteins are synthesized and form dimers. This dimer formation results in the activation of transcription of two reporter genes: the LexA-operator-Leu2 gene in the chromosome and the LexA-operator-LacZ reporter gene on the two (2) micron plasmid. As a result, yeast can grow on synthetic dropout medium without leucine and produce a blue color on medium containing XGal. When a library of potential TDNEs, i.e., cI-dfRas fusions, are over-expressed in this genetic background (from a two (2) micron plasmid) those cI-TDNE fusions proteins which efficiently interact with the AD-cRaf1 fusion result in the transcription activation of cI-operator-Lys2 reporter fusion. This activation allows yeast with the TDNE cI-dfRas fusions to be selected on dropout medium without lysine. A subset of the lysine-selected fusions may interact strongly with the AD-cRaf1 fusion and cause the complete loss (or modulation) of activation of the first two reporter genes, i.e., LexA-operator-Leu2 and LexA-operator-LacZ. Such fusions show both the “trap” and “blocking” phenotypes and are, therefore, candidate dominant-negative elements to be tested in tissue culture models for the reversal of the Ras transforming phenotype.

Many alternative embodiments are possible. For example in some embodiments dominant-negative elements can be directly selected by the “blocking” phenotype and may be selected in a non-fusion protein format.

This approach can be comprised of the following elements:

(1) cI-dfRas fusion gene on a two (2)-micron multicopy plasmid under the control of the ADH promoter region;

(2) AD-cRaf1 fusion gene on a two (2) micron plasmid under the control of a galactose inducible Gal1 promoter; and

(3) A library of dfRas DNA fragments on a two (2)-micron plasmid with antibiotic resistance gene.

When yeast SKY 191 cells are grown on galactose containing media both cI-dfRas and AD-cRaf1 are synthesized. An interaction between these fusion proteins causes the transcriptional activation of the cI-operator-Lys2 reporter gene in the chromosome of the yeast SKY191. Cell death would result when such cells are grown on media containing α-aminoadipate. The over-expression of a dominant-negative dfRas fragment in the same genetic background would diminish or abolish interaction between cI-dfRas and AD-cRaf1. The loss of this interaction leads to the loss of transcription activation of cI-operator-Lys2 reporter gene allowing yeast to grow on medium with α-aminoadipate. This strategy does not involve a fusion-based dominant-negative element. It does, however, allow the direct selection and identification of antisense sequences that could block expression of Ras and allow the identification of a different class of suppressor elements, in addition to protein-based dominant-negative elements.

The nucleic acids molecules of the invention can be present in vectors that result in the expression of these molecules over a wide range of levels. For example, in one embodiment, the LexA-dfRas fusion gene will be integrated in the chromosome of the SKY 191 strain employing an integrative vector to decrease the level of expression. Such modulation of expression of the LexA-dfRas fusion protein allows for controlled competition between LexA-dfRas and TDNE cI-dfRas proteins for interaction with AD-cRaf1.

The composition of the invention further includes libraries of TDNE comprising the cI DNA binding moiety covalently fused to the peptides derived from the proteins of interest. In addition similar libraries can be constructed with other DNA binding proteins appropriate to the host cells being employed.

The methods of the invention include preparation of TDNE libraries of the protein of interest. Such methods include, but are not limited to: the ultrasonic disruption to fragments in the size range of 200-700 base pairs, the use of randomly primed PCR products from the genes of interest, and the construction of families of fragments using an set of restriction nuclease treatments employing nucleases that show an array of four base pair sequence specificities.

The methods of the invention further include methods for identification of TDNE fragments, comprising: incubating an yeast cells of the invention, wherein a TDNE library of the first protein of interest expressed a long with two proteins of interest fused to DNA binding or to transcription activation domain. The interaction of TDNE with the activation domain fusion of the second protein of interest will cause the expression of specific reporter genes. The expression of these reporter genes will allow the growth of yeast cells of invention on specific synthetic dropout medium and will lead to isolation of TDNE from the protein of interest. In another method for TDNE identification, the expressed TDNE will modulate the interaction of the two proteins of interest. Such modulation will cause a transcriptional inactivation of the corresponding reporter gene and will lead to the growth of yeast cells of invention on specific synthetic dropout medium.

The methods of the invention further include the methods for identification of compounds, which modulate protein-protein interactions, for example the interaction between the TDNE and, the protein of interest. Such methods comprise the incubation of the yeast cells of invention and a test compound that disrupts the interaction between TDNE and the activation domain fusion of the second protein resulting in diminished expression of the cI-operator-Lys2 reporter gene. In instances where the yeast cells of the invention can grow in presence of tested compound on synthetic dropout medium containing α-aminoadipate. A compound that disrupts the interaction between the TDNE and the second protein of interest can be identified by growth on α-aminoadipate. In a second step, the effect of the identified compound on a protein-protein interaction of the first and second protein of interest may also be evaluated. In another method of compound identification, the activation of the cI-operator-LacZ reporter gene, resulting from an interaction between the TDNE and the activation domain fusion of the second protein of interest is assayed. In instances where the level of LacZ expression in yeast cells incubated with compound is lower than that found in control yeast cells incubated in the same growth medium without the test compound, an antagonist of the protein-protein interaction is identified. In instances where a greater level of LacZ reporter product expression is observed, an agonist of the protein-protein interaction is identified.

5.4.3. Identification of TDNEs Via E. coli AraC-Based Homodimer Detection Systems

E. coli can metabolize the pentose sugar arabinose by expressing the araBAD operon. However, enzymes encoded by araBAD are only produced when arabinose is present and the cells are in a low energy state. The regulatory protein AraC controls AraBAD expression.

AraC positively regulates the araBAD operon. This mode of regulation is accomplished by alternative binding of the AraC homodimer. In the absence of arabinose, the AraC dimer binds to two distinct sites in the ara operon. One subunit of the AraC protein contacts the araO₂ site, while the second subunit contacts the araI₁ site. This conformation causes DNA looping of the regulatory region which sterically interferes with the ability of RNA polymerase to interact with the promoter regions of both araC and araBAD. In the presence of arabinose, in contrast, the AraC homodimer dissociates from araO₂ and araI₁, and binds at the araI₁ and araI₂ half-sites. In this configuration the AraC protein activates transcription.

The AraC protein, which comprises 292 amino acid residues, consists of three (3) functional AraC domains. Stoner and Schleif, 1982, J. Mol. Biol. 152:649-652. Residues one (1) through 170 are involved in the dimerization of AraC molecules and the binding of arabinose. A flexible linker domain, i.e., amino acid residues 171 thorough 178, links the dimerization domain to the DNA binding domain and is defined by amino acid residues 179 through 292. The transcriptional activation function of the AraC protein is intimately associated with the DNA binding domain.

The modular AraC protein can be experimentally manipulated. For example, the linker region can be lengthened. Furthermore, the AraC dimerization domain may be replaced by a heterologous dimerization domains. In AraC chimera, where a heterologous domain provides the dimerization function, modulation of dimerization can be monitored by changes in AraC's transcriptional activation function. This function can be assayed via detection of AraC-dependent reporter gene expression. Any reporter gene sequences, e.g., reporter gene sequences as described above, and assays for detecting reporter gene expression, can be utilized as part of these assays.

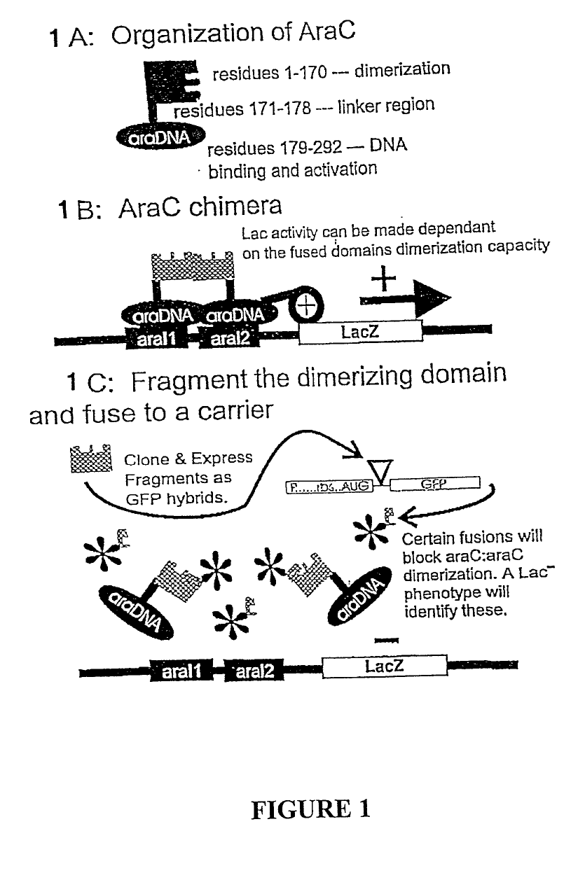

According to the present invention, cDNA sequences from, e.g., new genes of interest, either complete or partial, may be fused to the DNA binding/activation domain of AraC. The dimerization capacity of the so generated chimeras may be tested by measuring their ability to activate transcription of an AraC-dependent promoter, preferably operably linked to a reporter gene (see Section 5.4.1, supra). In those instances where activation is noted, libraries are generated which are composed of subfragments of the dimerizing sequences fused to a “carrier” protein, so that a TDNE library is generated. This AraC-based assay system is depicted in FIG. 1. Preferred carrier proteins include, but are not limited to GFP or GST in the bacterial homodimer interaction systems, and the C₁ protein in a modified dual bait 2-hybrid system. However, numerous other proteins or protein fragments may be preferred carriers depending on the specific experimental question to be resolved. The skilled artisan will be able to determine a most suitable carrier protein for any specific approach. Members of the library are subsequently tested for their ability to block the transactivation capacity of the AraC chimera. Those library members comprised of fragments with “blocking” activity will be candidate fusions appropriate for further testing as dominant-negative elements.

The above outlined approach based on the AraC system is particularly useful as a first test system for characterizing an unknown target protein. It is a very simple and inexpensive system which may be performed in E. coli an a very time efficient and high-throughput manner. This system allows to obtain relatively quickly insight into a gene products' oligomerization capacity. The AraC based system, generally, may be used to identify both heterodimeric and homodimeric protein-protein interactions, however, it is preferably used to identify and characterize homodimeruc protein-protein interaction. Even if applied to identify homodimeric interactions, the AraC based approach provides valuable information regarding the protein's tertiary structure in a very economical manner. Indeed, many known proteins show homodimerization, even proteins that undergo higher aggregation states involving multi-subunit associations often show a homodimeric component.

The AraC based analysis of homodimeric interactions can be undertaken as soon as a new sequence, or partial sequence is known. The analysis of higher order multi-subunit associations may require identification of the other potential interacting components. The analysis of a new protein for homodimeric interaction-based dominant-negative fusions can be completed as potential interacting proteins are identified utilizing techniques like, e.g., the yeast two-hybrid system. See, infra.

TDNEs which block a homodimeric interaction in the test AraC system can be expected to block homodimeric interactions of the corresponding target protein, that is not fused to AraC, in its native context. Blocking TDNEs can, therefore, be expected to disrupt the native tertiary structure of its cognate parental protein. This is expected to lead to the ablation of function. Ablation of function will allow to test any hypothesized function, moreover, it may even lead to the discovery of the protein's function, even in the absence of a hypothesis. In addition to providing a TDNE that can assess the function of a target protein of interest in model systems, see, infra, such a dominant-negative fusion protein may also have value as a therapeutic agent in those cases where the ablation of function has desirable therapeutic endpoint. Further, in such cases where the TDNE identified in the AraC based assay system exhibits dominant-negative activity in functional test systems, e.g., tissue culture systems or in vivo systems, see, infra, the same AraC based system as originally employed may be used to identify small molecules which block the same protein-protein interaction that initially uncover the inhibitory TDNE. Such a small molecule could provide the starting point for the development of therapeutic compounds, including small molecule compounds, or their leads.

### 5.5. Assay Systems For The Verification Of The Dominant-Negative Activity

The assay system strategies described herein are designed to identify TDNEs which have the capacity to block a protein-protein interaction. Such a protein-protein interaction and/or TDNEs that modulate such a protein-protein interaction can be identified in microbial systems independent of any knowledge about the protein's function. The identified protein-protein interactions and the TDNEs identified via such microbial systems can then be verified and further characterized by addressing the function of the protein-protein interaction and/or the activity or result of expressing the TDNE in an environment native to that of the target protein of interest (that is, a cellular environment in which the target protein is normally expressed and and the protein-protein interaction of interest is normally found). Methods for accomplishing this verification are described herein.

In one embodiment, where the target gene/protein of interest is mammalian, e.g., a human gene/protein, test systems based on mammalian or human cells can be employed. Standard mammalian transfection, expression, including regulatable expression, can be utilized for such methods. In another embodiment, the test protein may be viral, fungal or bacterial in origin, and tested in the native host cell type of the target protein. A system suitable for determination of the significance of a protein-protein interaction to a protein's in vivo function has to be designed such that “blocking” will disrupt the interactions of the native protein leading to the ablation of native function. Interpreting the consequences of loss of function resulting from the expression of TDNE will in turn be used to aid in defining the protein's function.

Tissue Culture Based Systems. The experimentally most tractable systems for testing mammalian target proteins of interest will be mammalian tissue culture cells, although one can envision expression in transgenic animal models. Expression in these systems is well known to anyone practiced in the art.

In preferred, but non-limiting embodiments, expression should be subject to regulation. Regulated expression will allow the controlled evaluation of potential phenotypes that could involve lethality in the case of essential genes. In mammalian cells the Tet-on expression plasmids developed by Bjuard provide the backbone for the most preferred embodiments. Gossen et al., 1995, Science 268:1766-1769. In this system a blocking chimera is subcloned under control of a tetracycline inducible promoter, stable transfectants are established in a cell line expressing the Tet-on activator, and a phenotype in the native environment is evaluated following tetracycline-induced expression of the interaction blocking chimera.

Bacterial Assays. For testing target proteins that are native to bacterial or other microbial cells, the appropriate bacterial or microbial host cells will be used to test the activity of the target protein of interest. For example, in one embodiment, the assays described herein may be used to identify molecules that interrupt critical protein-protein interactions required for bacterial cell growth, to be used, for example, as antibiotic compounds. In this case, it is preferable that a yeast-based protein-protein interaction system is used to identify a candidate TDNE, since an inhibitor of an important protein-protein interaction of a critical bacterial protein would likely be toxic to its bacterial host. Once candidate TDNEs are identified, expression in the target protein's native bacterial host could be used to test the activity and potency of the TDNE. Methods for protein expression and analysis in bacteria described in Section 5.4.1, supra, can be used for expression and analysis of candidate TDNEs in their native microbial host cells.

Transgenic Animals Overexpressing The TDNE Of Interest. In one embodiment of the invention, TDNEs identified to affect a target protein's protein-protein interactions are further tested in transgenic animals. Transgenic animals overexpressing the selected TDNE are generated using methods generally known in the art. Numerous strategies for producing genetically engineered animals are known in the art, including DNA microinjection, embryonic stem cell- or retrovirus-mediated transfer. General protocols and vector systems suitable for the generation of transgenic animals overexpressing a selected TDNE can be found, e.g., in Transgenic Animal Technology: A Laboratory Handbook, 1994, ed: Pinkert, C. A. Academic Press, Inc., San Diego, Calif. In preferred embodiments, mice are used as host animals, but numerous other species may be used for the generation of transgenics, including, but not limited to, cows, rats, poultry, fish, goat, sheep, and swine.

Generation And Use Of Antibodies To Aid Identification Of The Target Protein's Function. As described in more detail, infra, antibodies directed to the identified dominant-element may be generated. Since these antibodies are by nature directed to a “binding” domain of the target protein, they are likely to exhibit inhibitory effects on the function of the target protein. These antibodies, therefore, may be used in tissue culture or in vivo to aid and/or confirm the target protein's biological function. Such antibodies should lead to essentially the same result as similar approaches using the identified TDNE's or dominant-negative elements. See, supra.

5.5.1. Generation And Use Of Antibodies Directed To The Identified Dominant-Negative Elements

Various procedures known in the art may be used for the production of antibodies to epitopes of the identified dominant-negative elements identified and isolated employing systems of the present invention. Such antibodies include but are not limited to polyclonal, monoclonal, chimeric, single chain, Fab fragments and fragments produced by an Fab expression library. Such antibodies may be useful, e.g., as diagnostic or therapeutic agents, or as agents for the identification of the target protein's function in vivo, see, supra.

The generation of antibodies using the dominant-negative elements identified with the methods of the present invention is particularly useful when the generation of neutralizing antibodies is the objective, for example as therapeutic agent. This is true as antibodies directed against the dominant-negative element can be expected to act neutralizing, i.e., to interfere with the target protein's activity.

For use as diagnostic agents, monoclonal antibodies that bind to the dominant-negative element and thus to the target protein of interest are radioactively labeled to allow detection of their location and distribution in the body after injection. Radioactivity tagged antibodies may be used as a non-invasive diagnostic tool for imaging in vivo the presence of a tumors and metastases associated with the expression of the target protein.

Immunotoxins may also be designed which target cytotoxic agents to specific sites in the body. For example, high affinity monoclonal antibodies may be covalently complexed to bacterial or plant toxins, such as diphtheria toxin, abrin, or ricin. A general method of preparation of antibody/hybrid molecules may involve use of thiol-crosslinking reagents such as SPDP, which attack the primary amino groups on the antibody and by disulfide exchange, attach the toxin to the antibody. The hybrid antibodies may be used to specifically eliminate cells expressing the target protein, which may be particularly desired if the target protein is associated with a disease related to unregulated cell proliferation such as cancer.

For the production of antibodies, various host animals are immunized by injection with the dominant-negative element including, but not limited to, rabbits, mice, rats, etc. For the purpose of antibody production, the non-specific part, e.g., the cI, of the TDNEs is removed, such that only the dominant-negative element is used for immunization. The dominant-negative element may be fused to a non-immunogenic carrier protein in order to increase its stability. Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanin, dinitrophenol, and potentially useful human adjuvants such as BCG (bacille Calmette-Guerin) and Corynebacterium parvum.

Monoclonal antibodies to the dominant-negative element may be prepared using any technique which provides for the production of antibody molecules by continuous cell lines in culture. These include, but are not limited to, the hybridoma technique originally described by Kohler and Milstein, 1975, Nature 256:495-497, the human B-cell hybridoma technique (Kosbor et al., 1983, Immunology Today 4:72; Cote et al., 1983, Proc. Natl. Acad. Sci. U.S.A. 80:2026-2030) and the EBV-hybridoma technique (Cole et al., 1985, Monoclonal Antibodies and Cancer Therapy, Alan R. Liss, Inc., pp. 77-96). In addition, techniques developed for the production of “chimeric antibodies” (Morrison et al., 1984, Proc. Natl. Acad. Sci. U.S.A. 81:6851-6855; Neuberger et al., 1984, Nature 312:604-608; 25 Takeda et al., 1985, Nature 314:452-454) by splicing the genes from a mouse antibody molecule of appropriate antigen specificity together with genes from a human antibody molecule of appropriate biological activity can be used. Alternatively, techniques described for the production of single chain antibodies (U.S. Pat. No. 4,946,778) can be adapted to produce single chain antibodies specific for the dominant-negative element.

Antibody fragments which contain specific binding sites of the dominant-negative element may be generated by known techniques. For example, such fragments include, but are not limited to, F(ab′)₂ fragments which can be produced by pepsin digestion of the antibody molecule and the Fab fragments which can be generated by reducing the disulfide bridges of the F(ab′)₂ fragments. Alternatively, Fab expression libraries may be constructed (Huse et al, 1989, Science 246:1275-1281) to allow rapid and easy identification of monoclonal Fab fragments with the desired specificity to the dominant-negative element.

### 5.6. Use Of Tag Dominant-negative Elements For Identification Of Gene Function

The use of TDNEs can be directed towards exploring the function of known, well defined genes and the products they encode. Alternatively, TDNEs can be used to define and describe the function of genes and their respective protein products which are poorly or not at all understood. This application is particularly important in light of the recent advances in genomics and the discovery that a large number of genes uncovered during genome sequencing products have no known homologs and therefore their functions cannot be inferred. Several general approaches will be employed to elucidate gene function using TDNEs.

### 5.7. Use Of Isolated Dominant-negative Elements For The Identification Of Compounds Useful For The Treatment Of Disease Related To The Target Protein

TDNEs and peptide sequences identified using the methods of the invention may be used directly in gene therapy methods to modulate protein-protein interactions of particular interest. In addition, TDNEs generated using the methods of the invention may be further modified to select for desired properties. Depending on the end application certain biochemical properties of the TDNEs may be altered including binding affinities, binding specificities, stability, therapeutic affects, etc (Moore and Arnold, 1996, Nat. Biotech. 14:458-467). Further, TDNEs may be used to screen for other compounds, e.g., small molecules, that interfere with the protein-protein interaction of interest. The methods described herein describe methods that can be used to identify novel compounds useful as therapeutics.

5.7.1. TDNEs with Improved Specificity

Specific properties of the TDNE can be modified by subjecting the gene to random mutagenesis to create a sub-library of TDNEs that can be utilized in a subsequent round of assays or screens designed to identify changes in the desired property. This process can be iterated numerous times to optimize the property of interest. This technique has been referred to as directed evolution (Shao et al., 1998, Nucleic Acids Res 26:681-683). Changes in the biochemical properties of the parental TDNEs can be efficiently monitored using the assay systems described in the invention or other screens may be utilized that are more suited to identifying a change in a specific property of the TDNE. For example, a TDNE may be identified as a candidate therapeutic agent. A better candidate may be desired that has an increased binding affinity for its receptor/partner. An increase in its binding affinity could be identified by randomly mutating its gene sequence to create a sub-library of TDNEs. This sub-library of TDNE is employed in the assays described herein allows for the rapid identification of improved binding affinity of a specific a TDNE by simple colormetric or flourometric techniques.

Various techniques are known for replicating specific DNA sequences in such a manner as to produce errors i.e. variants in the DNA sequence. These methods are easily accessible to the skilled practioner and including but not limited to error prone PCR (Cline et al., 1996, Nucleic Acids Res. 24: 3546-3551), DNA family shuffling (Christians et al., 1999, Nat. Biotech. 17:259-264) combinatorial mutagenesis (MacBeath et al., 1998, Prot. Sci. 7:1757-1767). These methods have been used to alter specific biochemical properties by 4-5 orders of magnitude and provide a powerful method for improving the activity of TDNEs identified by the means of the invention.

5.7.2. Drug Design

TDNEs can also be used to design novel drugs and small molecule therapeutics, based on the knowledge of the TDNE peptide sequence. For example, the three dimensional structure of the TDNE can be determined, and the active site of a TDNE-target protein-protein interaction can be modeled. This can be done by known methods, including X-ray crystallography, which can determine a complete molecular structure. On the other hand, solid or liquid phase NMR can be used to determine certain intra-molecular distances. Any other experimental method of structure determination can be used to obtain partial or complete geometric structures. The geometric structures of target protein may be measured with a complexed TDNE ligand, which may increase the accuracy of the active site structure determined.

If an incomplete or insufficiently accurate structure is determined, the methods of computer based numerical modeling can be used to complete the structure or improve its accuracy. Any recognized modeling method may be used, including parameterized models specific to particular biopolymers such as proteins or nucleic acids, molecular dynamics models based on computing molecular motions, statistical mechanics models based on thermal ensembles, or combined models. For most types of models, standard molecular force fields, representing the forces between constituent atoms and groups, are necessary, and can be selected from force fields known in physical chemistry. The incomplete or less accurate experimental structures can serve as constraints on the complete and more accurate structures computed by these modeling methods.

Finally, having determined the structure of the active site, either experimentally, by modeling, or by a combination, candidate modulating compounds can be identified by searching databases containing compounds along with information on their molecular structure. Such a search seeks compounds having structures that match the determined active site structure and that interact with the groups defining the active site. Such a search can be manual, but is preferably computer assisted. These compounds found from this search are potential compounds that blocks the function of the ribosomal protein of interest.

Using these methods, the composition of a TDNE can be modified and the structural effects of modification can be determined using the experimental and computer modeling methods described above applied to the new composition. The altered structure can then be compared to the TDNE active site structure to determine if an improved fit or interaction results. In this manner systematic variations in composition, such as by varying side groups, can be quickly evaluated to obtain inhibitory compounds of improved specificity or activity.

Further experimental and computer modeling methods useful to identify compounds based upon identification of the protein-protein interaction site or surface of the target protein will be apparent to those of skill in the art. Examples of molecular modeling systems are the CHARMm and QUANTA programs (Polygen Corporation, Waltham, Mass.). CHARMm performs the energy minimization and molecular dynamics functions. QUANTA performs the construction, graphic modeling and analysis of molecular structure. QUANTA allows interactive construction, modification, visualization, and analysis of the behavior of molecules with each other.

A number of articles review computer modeling of drugs interactive with specific proteins, such as Rotivinen et al. (1988, Acta Pharmaceutical Fennica 97:159-166); Ripka (1988 New Scientist 54-57); McKinaly and Rossmann (1989, Annu. Rev. Pharmacol. Toxiciol. 29:111 -122); Perry and Davies, OSAR: Quantitative Structure-Activity Relationships in Drug Design pp. 189-193 Alan R. Liss, Inc. 1989; Lewis and Dean (1989, Proc. R. Soc. Lond. 236:125-140 and 141-162); and, with respect to a model receptors for nucleic acid components, Askew, et al. (1989, J. Am. Chem. Soc. 111: 1082-1090). Other computer programs that screen and graphically depict chemicals are available from companies such as BioDesign, Inc. (Pasadena, Calif.), Allelix, Inc. (Mississauga, Ontario, Canada), and Hypercube, Inc. (Cambridge, Ontario).

5.7.3. A Protein with Multiple Interaction Domains Can Be Dissected into Individual Domains Providing Drug Discovery Opportunities Not Available with Full-Length Proteins

The division of a large interaction surface into constituent parts provides opportunities for drug discovery processes. While it might be difficult to directly identify a small molecule drug discovery lead that can block a large interaction surface, a molecule coan be found that blocks the smaller constituent components of the larger interaction. In a combinatorial process smaller molecules found to block components of the larger protein-protein interaction can be covalently linked to produce a larger molecule that blocks the larger complete interaction surface. The systems used to confirm the constituents interactions (e.g., an ELISA signal) can be employed to identify small molecules that block those smaller constituents interactions by screening for loss of that interaction signal. In this paradigm it is possible to incrementally arrive at a molecule that blocks a larger interaction that is composed of the constituent subdomains.

For example, as demonstrated below, in Section 6.5, the smallest piece of the TNFα receptor extracellular domain (TNFαR ECD) found to compete is the 95 amino acid C77-172 piece. Naismyth and Spanger (infra) have described much smaller modules (“A” modules, 12-17 residues, and “B” modules 21-24 residues) which occur as common structural subunits across the entire TNFα receptor family. The C77-172 piece is composed of several such units and it is reasonable to expect that the C77-172 piece can be further subdivided into smaller functional subunits. This is accomplished by designing specific PCR primers that could be used to form MalE fusions specifically designed to contain the constituent modules. Alternatively more random approaches such as, for example, the CviJI strategy of Example 4, below, (or others described herein) can be employed to produce random libraries. Either type of library can be screened for members that are able to compete with the full ECD of the CadC-TNFαR construct and to identify the constituent modules involved in defining the protein-protein interaction.

Once such competing modules are identified, their ability to interact with the full-length TNFαR ECD can be tested in a variety ways. The full-length TNFαR can, e.g., be cloned as a chimera of the LexA (or CI) DNA binding domain in the systems described in Examples 3 and 4, below, while the candidate subdomains can be tested by fusion to the activating domain. The observation of a signal in the system provides confirmation that the domains shown to block in the CadC-based system in fact do so by physically interacting. Alternatively the interactions could be tested by direct physical techniques such as an ELISA. (The MalE portion of the MalE fusions would aid in the preparation of the required ELISA reagents.)

5.7.4. Identification Of Compounds

Two general types of assay systems are used for the identification and isolation of compounds, including small molecule compounds, peptides etc., inhibiting the function of the target protein of interest, which has been identified to have a specific biological function and/or is involved in the development of a type of disease. First, assay systems are employed which measure the ability of a compound to interfere with the protein-protein interaction itself, i.e., the measured parameter is interference of the compound with the binding of the protein to its partner. A number of suitable assay systems can be envisioned. These assay systems may either measure the “binding” interaction of the full-length target protein, fragments thereof. However, typically, interference of a compound with binding of the identified TDNEs (or isolated dominant-negative element) with the target protein will be measured. For example, systems such as the araC based assay system, the CadC-based system or the modified dual bait-based system, expressing the target protein and the identified TDNE may be directly employed for the identification of compounds inhibiting their protein-protein interaction. See, supra. Such assays are particularly suitable for high-throughput screening of test compounds and for the identification of leads. Secondly, assay systems may be used which are tailored to the specific type of target protein and its function. For example, in vitro and in vivo assays may be developed which measure inhibition of a specific biological function of the target protein of interest, which has been determined using the above described assays and animal models. This second general type of assays does not require a co-expression of the identified TDNEs, since interference of the chemical compounds with the biological function of the target protein is directly measured.

The first type of assay system is based on the same concept as the assay systems for the identification and isolation of dominant-negative elements. See, supra. If used for compound screening, however, the assay system is designed such that two identified binding partners, e.g., the target protein X and the previously identified TDNE, see, supra, are each fused to one of the DNA-binding domain (DB) or the activation domain (AD) of the assay system, respectively. Alternatively, two full-length target proteins, or any kind of fragment thereof which comprises the site which is required for their interaction, may be fused to DB and AD. Cells expressing both DB- and AD fusions are exposed to libraries of compounds, e.g., small molecule compounds (Gallop et al., 1994, J. Med. Chem. 37:1233-1251), and interruption of the protein-protein interaction is measured by suppression of the reporter gene expression.

The second type of assay system includes any kind of in vitro or in vivo system which allows identification of compounds which interfere with the biological activity of the target protein. The design of such an assay is of course dependent on the type of target protein and on the type of its biological activity. For example, if the target protein is a regulator of cell proliferation, the effect of test compounds on cell proliferation may be measured in a suitable system. If the target protein is a promoter of cell proliferation, inhibition of proliferation may, e.g., be measured in cell culture ³H-Thymidine assays (Detrick-Hooks et al., 1975, J. Immunol. 114:287-290; Dean et al., 1977, Int. J. Cancer 20:359-370), transformation assays (Pettengill et al., 1980, Ex. Cell. Biol. 48:279-297; Kahn et al., 1979, J. Cell. Biol. 82:1-16, or soft agar colony formation assays (Schlag and Flentje, 1984, Cancer Treat. Rev. 11 Suppl A:131-137; Osborne et al., 1985, Breast Cancer Res. Treat. 6:229-235).

If the target protein has been shown to have a particular enzymatic activity, e.g., kinase activity, inhibition of this enzymatic activity can be measured in vitro or in vivo using radiolabelled substrate or suitable antibodies for measuring enzymatic activity. Preferably, these assay systems are designed that they allow for high-throughput testing of compounds from any source to identify molecules having the desired effect on the biological function of the target protein. In many cases, assay systems measuring the effects of chemical compounds on the biological function of the target protein will be performed as a “second” step, using positive hits identified with high-throughput systems measuring the effect of test compounds on the protein-protein interactions which have been determined using the TDNE libraries of the invention.

Nucleotide sequences encoding the TDNEs, dominant-negative elements and target proteins identified and isolated using assay systems of the invention may be used to produce the corresponding purified protein using well-known methods of recombinant DNA technology. Among the many publications that teach methods for the expression of genes after they have been isolated is Gene Expression Technology, Methods and Enzymology, Vol.: 185, edited by Goeddel, Academic Press, San Diego, Calif. (1990).

The nucleotide acid molecules may be expressed in a variety of host cells, either prokaryotic or eukaryotic. In many cases, the host cells would be eukaryotic, more preferably host cells would be mammalian. Host cells may be from species either the same or different than the species from which the target protein is naturally present, i.e., endogenous. Advantages of producing the target protein by recombinant DNA technology include obtaining highly enriched sources of the proteins for purification and the availability of simplified purification procedures. Methods for recombinant production of proteins are generally very well established in the art, and can be found, among other places in Sambrock et al., 1990, Molecular Cloning: A Laboratory Manual, Cold Spring Harbour Laboratory Press, New York.

In one embodiment of the invention, cells transformed with expression vectors encoding the target protein and/or corresponding TDNEs or dominant-negative elements are cultured under conditions favoring expression of their expression and the recovery of the recombinantly-produced protein from the cell culture. A target protein, TDNE or dominant-negative element produced by a recombinant cell may be secreted or may be contained intracellularly, depending on the nature of the target protein and the particular genetic construction used. In general, it is more convenient to prepare recombinant proteins in secreted form. Purification steps will depend on the nature of the production and the particular target protein, TDNE or dominant-negative element produced. Purification methodologies are well established in the art; the skilled artisan will know how to optimize the purification conditions. General protocols of how to optimize the purification conditions for a particular protein can be found, among other places, in Scopes in: Protein Purification: Principles and Practice, 1982, Springer-Verlag New York, Heidelberg, Berlin.

In addition to recombinant production, peptide fragments may be produced by direct peptide synthesis using solid-phase techniques. See, Stewart et al., Solid-Phase Peptide Synthesis (1969), W. H. Freeman Co., San Francisco; and Merrifield, 1963, J. Am. Chem. Soc. 85:2149-2154.

In vitro polypeptide synthesis may be performed using manual techniques or by automation. Automated synthesis may be achieved, for example, using Applied Biosystems 431A Peptide Synthesizer (Foster City, Calif.) following the instructions provided in the instruction manual supplied by the manufacturer.

In one embodiment of the invention, the target protein, TDNE or dominant-negative element and/or expressing cell lines expressing the same are used to screen for antibodies, peptides, organic molecules or other ligands that act as agonist or antagonists of their biological activity. For example, antibodies capable of interfering with the activity, e.g., enzymatic activity of the target protein, or with its interaction with a ligand, adapter molecule, or substrate are used to inhibit its biological function. In cases where amplification of the target protein's function is desired, antibodies which mimic, e.g., a ligand, an adapter molecule or substrate of the corresponding the signal transduction pathway may be developed. Obviously, if desired, antibodies may be generated which modify the activity, function, or specificity of the target protein.

Alternatively, screening of peptide libraries or small molecule organic compounds with recombinantly expressed target protein, TDNE or dominant-negative element or cell lines expressing the same may be useful for identification of therapeutic molecules that function by inhibiting, enhancing, or modifying its biological activity.

Synthetic compounds, natural products, and other sources of potentially biologically active materials can be screened in a number of ways. The ability of a test compound to inhibit, enhance or modulate the function of the target protein may be determined with suitable assays measuring the target protein's function. For example, responses such as its activity, e.g., enzymatic activity, or its ability to bind its ligand, adapter molecule or substrate may be determined in in vitro assays. Cellular assays may be developed to monitor a modulation of second messenger production, changes in cellular metabolism, or effects on cell proliferation. These assays may be performed using conventional techniques developed for these purposes. Finally, the ability of a test compound to inhibit, enhance or modulate the function of the target protein will be measured in suitable animal models in vivo. For example, mouse models will be used to monitor the ability of a compounds to inhibit the development of solid tumors, or effect reduction of the solid tumor size.

In one embodiment of the invention, random peptide libraries consisting of all possible combinations of amino acids attached to a solid phase support are used to identify peptides that are able to interfere with the function of the target protein. For example, peptides may be identified binding to a ligand-, adapter molecule- or substrate binding site of a given target protein or other functional domains of the target protein, such as an enzymatic domain. Accordingly, the screening of peptide libraries may result in compounds having therapeutic value as they interfere with its activity.

Identification of molecules that are able to bind to the target protein may be accomplished by screening a peptide library with recombinant soluble target protein, TDNE or dominant-negative element. Methods for expression and purification of the selected cell proliferation genes are generally known in the art (see, Sambrock et al., 1994, Molecular Cloning: A Laboratory Manual, Cold Spring Harbor Press, New York; Ausubel et al., Current Protocols in Molecular Biology, Greene Publishing Associates and Wiley Interscience, N.Y.), and may be used to express recombinant full length cell target protein or fragments thereof, depending on the functional domains of interest.

In order to identify and isolate the peptide/solid phase support that interacts and forms a complex with the target protein, TDNE or dominant-negative element, it is necessary to label or “tag” the same or fragment thereof. For example, the target protein, TDNE or dominant-negative element may be conjugated to enzymes such as alkaline phosphatase or horseradish peroxidase or to other reagents such as fluorescent labels which may include fluorescein isothyiocynate (FITC), phycoerythrin (PE) or rhodamine. Conjugation of any given label to the target protein, TDNE or dominant-negative element may be performed using techniques that are routine in the art.

In addition to using soluble target protein molecules or fragments thereof for the identification of binding partners, in another embodiment, peptides that bind to the target protein may be identified using intact cells. The use of intact cells is preferred for use with target protein which comprise cell surface receptors, which require the lipid domain of the cell membrane to be functional. Methods for generating cell lines expressing the target proteins, TDNEs or dominant-negative elements identified my means of the invention are generally known in the art and can be found in e.g., Sambrock et al., 1994, Molecular Cloning: A Laboratory Manual, Cold Spring Harbor Press, New York; and Ausubel et al., Current Protocols in Molecular Biology, Greene Publishing Associates and Wiley Interscience N.Y. The cells used in this technique may be either live or fixed cells. The cells are incubated with the random peptide library and will bind to certain peptides in the library. The so formed complex between the target cells and the relevant solid phase support/peptide may be isolated by standard methods known in the art, including differential centrifugation.

As an alternative to whole cell assays for membrane bound receptors or receptors that require the lipid domain of the cell membrane to be functional, the receptor molecules can be reconstituted into liposomes where a label or “tag” can be attached.

In another embodiment, cell lines that express the target protein or fragments thereof, are used to screen for molecules that inhibit, enhance, or modulate the target protein's activity or signal transduction. Such molecules may include small organic or inorganic compounds, or other molecules that effect the target protein's activity or that promote or prevent the complex formation with its ligand, adapter molecules, or substrates. Synthetic compounds, natural products, and other sources of potentially biologically active materials can be screened in a number of ways, which are generally known by the skilled artisan.

For example, the ability of a test molecule to interfere with the target protein's function may be measured using standard biochemical techniques. Alternatively, cellular responses such as activation or suppression of a catalytic activity, phosphorylation or dephosphorylation of other proteins, activation or modulation of second messenger production, changes in cellular ion levels, association, dissociation or translocation of signalling molecules, or transcription or translation of specific genes may also be monitored. These assays may be performed using conventional techniques developed for these purposes in the course of screening.

Further, effects on the target protein's function may, via signal transduction pathways, affect a variety of cellular processes. Cellular processes under the control of the target protein's signalling pathway may include, but are not limited to, normal cellular functions, proliferation, differentiation, maintenance of cell shape, and adhesion, in addition to abnormal or potentially deleterious processes such as unregulated cell proliferation, loss of contact inhibition and, blocking of differentiation or cell death. The qualitative or quantitative observation and measurement of any of the described cellular processes by techniques known in the art may be advantageously used as a means of scoring for signal transduction in the course of screening.

Various technologies may be employed for the screening, identification, and evaluation of compounds which interact with the target protein of the invention, which compounds may affect various cellular processes under the control of said target protein.

For example, the target protein or a functional derivative thereof, in pure or semi-pure form, in a membrane preparation, or in a whole live or fixed cell is incubated with the compound. Subsequently, under suitable conditions, the effect of the compound on the target protein's function is scrutinized, e.g., by measuring its activity, or its signal transduction, and comparing the activity to that of target protein, incubated under same conditions, without the compound, thereby determining whether the compound stimulates or inhibits the target protein's activity.

In addition to the use of whole cells expressing the target protein, TDNE or dominant-negative element for the screening of compounds, the invention also includes methods using soluble or immobilized target protein, TDNE or dominant-negative element. For example, molecules capable of binding to the target protein may be identified within a biological or chemical preparation. For example, the target protein, or functional fragments thereof, e.g., fragments containing a specific domain of interest, is immobilized to a solid phase matrix, subsequently a chemical or biological preparation is contacted with the immobilized target protein for an interval sufficient to allow the compound to bind. Any unbound material is then washed away from the solid phase matrix, and the presence of the compound bound to the solid phase is detected, whereby the compound is identified. Suitable means are then employed to elute the binding compound.

5.7.5. Source Of Candidate Test Compounds

The test compounds employed for such assays are obtained from any commercial source, including Aldrich (1001 West St. Paul Ave., Milwaukee, Wis. 53233), Sigma Chemical (P.O. Box 14508, St. Louis, Mo. 63178), Fluka Chemie AG (Industriestrasse 25, CH-9471 Buchs, Switzerland (Fluka Chemical Corp. 980 South 2nd Street, Ronkonkoma, N.Y. 11779)), Eastman Chemical Company, Fine Chemicals (P.O Box 431, Kingsport, Tenn. 37662), Boehringer Mannheim GmbH (Sandhofer Strasse 116, D-68298 Mannheim), Takasago (4 Volvo Drive, Rockleigh, N.J. 07647), SST Corporation (635 Brighton Road, Clifton, N.J. 07012), Ferro (111 West Irene Road, Zachary, La. 70791), Riedel-deHaen Aktiengesellschaft (P.O. Box D-30918, Seelze, Germany), PPG Industries Inc., Fine Chemicals (One PPG Place, 34th Floor, Pittsburgh, Pa. 15272). Further any kind of natural products may be screened using the assay cascade of the invention, including microbial, fungal or plant extracts.

Furthermore, combinatorial libraries of test compounds, including small molecule test compounds may be commercially obtained from Specs and BioSpecs B.V. (Rijswijk, The Netherlands), Chembridge Corporation (San Diego, Calif.), Contract Service Company (Dolgoprudny, Moscow Region, Russia), Comgenex USA Inc. (Princeton, N.J.), Maybridge Chemicals Ltd. (Cornwall PL34 OHW, United Kingdom), and Asinex (Moscow, Russia), or may be generated as disclosed in Eichler and Houghten, 1995, Mol. Med. Today 1:174-180; Dolle, 1997, Mol. Divers. 2:223-236; Lam, 1997, Anticancer Drug Des. 12:145-167. These references, incorporated hereby by reference in their entirety, also teach additional screening methods which may be employed for the identification and isolation of leads and therapeutic compounds having a desired effect on the physiological activity and/or function of the target protein of interest, as identified according to the methods of the present invention.

5.7.6. Indications For The Use Of Compounds Interfering With The Protein/Protein Interactions Identified Using The Assays Of The Invention

The compounds identified by the above methods are modulators of the activity of a target protein of interest in general. As such, the compounds produced by the processes and assays of the invention are useful for the treatment of disease related to aberrant, uncontrolled or inappropriate function or activity of the target protein. The methods of the present invention for identifying dominant-negative elements inhibiting the function of a target protein, thereby facilitating elucidation of its biological function and role in disease development and manifestation, is not limited to any particular type or group of target proteins. Depending of the physiological role of each specific target protein, the compounds of the present invention are useful for the treatment of a variety of different types of diseases, including, but not limited to metabolic disorders, cell proliferative disorders, endocrine disorders, dysfunction of central or peripheral nervous systems, infection diseases both bacterial and infectious and inflammatory diseases.

For example, if the target protein is involved in the regulation of metabolic processes, such as amino acid synthesis, post-transcriptional processing of enzymes and other cellular components, respiratory or digestive processes, cardiovascular and neurological processes, bone formation processes, immunological and other inflammatory processes, and other processes affecting the metabolism of organs and tissues, the identified therapeutic compound will be used for the treatment of metabolic disorder, including, but not limited to, diabetes mellitus, plenylketonuria, ADA, porphyria, depression, Alzheimer's disease, aging, obesity, sleep disorders, cutaneous disorders, arrhythmias, renal disorders, visual disorders, and auditory disorders.

In other instances, the target protein is involved in the regulation of cell proliferation. A large number of disease states involve excess or diminished cell proliferation. Generally, many of these diseases may be treated with DNA sequences, proteins, or small molecules that influence cell proliferation. In some instances the goal is to stimulate proliferation; in others, to prevent or inhibit proliferation of cells. The list of diseases directly involving cell growth includes, but is not limited to, cancer, psoriasis, inflammatory diseases, such as rheumatoid arthritis, restenosis, immunological activation or suppression, including tissue rejection, neurodegeneration or expansion of neuronal cells and viral infection.

Accordingly, pharmaceutical compositions comprising a therapeutically effective amount of a compound identified by the methods of the invention will be useful for the treatment of diseases driven by metabolic dysfunction, including, but not limited to diabetes mellitus, plenylketonuria, ADA, porphyria, depression, Alzheimer's disease, aging, obesity, sleep disorders, cutaneous disorders, arrhythmias, renal disorders, visual disorders, and auditory disorders., or unregulated or inappropriate cell proliferation, including, but not limited to, cancer, such as glioma, melanoma, Kaposi's sarcoma, psoriasis, hemangioma and ovarian, breast, lung, pancreatic, prostate, colon and epidermoid cancer, rheumatoid arthritis, psoriasis, restenosis, immunological activation or suppression, including tissue rejection, neurodegeneration or expansion of neuronal cells.

### 5.8. Formulations/Route Of Administration

The identified compounds can be administered to a human patient alone or in pharmaceutical compositions where they are is mixed with suitable carriers or excipient(s) at therapeutically effective doses to treat or ameliorate a variety of disorders. A therapeutically effective dose further refers to that amount of the compound sufficient to result in amelioration of symptoms as determined in accordance with the symptoms of the particular disease. For example, in the case of a cancer treatment, amelioration of symptoms is determined by decrease of cellular growth. Techniques for formulation and administration of the compounds of the instant application may be found in “Remington's Pharmaceutical Sciences,” Mack Publishing Co., Easton, Pa., latest edition.

5.8.1. Routes Of Administration

Suitable routes of administration may, for example, include oral, rectal, transmucosal, or intestinal administration; parenteral delivery, including intramuscular, subcutaneous, intramedullary injections, as well as intrathecal, direct intraventricular, intravenous, intraperitoneal, intranasal, or intraocular injections.

Alternately, one may administer a compound of the invention in a local rather than systemic manner, for example, via injection of the compound directly into a solid tumor, often in a depot, or in a sustained release formulation.

Furthermore, one may administer the drug via a targeted drug delivery system, for example, in a liposome coated with tumor-specific antibody. The liposomes will be targeted to and taken up selectively by the tumor.

5.8.2. Composition/Formulation

The pharmaceutical compositions of the present invention may be manufactured by means of conventional mixing, dissolving, granulating, dragee-making, levigating, emulsifying, encapsulating, entrapping or lyophilizing processes.

Pharmaceutical compositions for use in accordance with the present invention thus may be formulated in a conventional manner using one or more physiologically acceptable carriers comprising excipients and auxiliaries which facilitate processing of the active compounds into preparations which can be used pharmaceutically. Proper formulation is dependent upon the route of administration chosen.

For injection, the agents of the invention may be formulated in aqueous solutions, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers well known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained as a solid excipient, optionally grinding a resulting mixture, and processing the mixture of granules, after adding suitable auxiliaries, if desired, to obtain tablets or dragee cores. Suitable excipients include fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with fillers such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

For buccal administration,the compositions may take the form of tablets or lozenges formulated in conventional manner.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin, for use in an inhaler or insufflator, may be formulated containing a powder mix of the compound and a suitable powder base such as lactose or starch.

The compounds may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Alternatively, the active ingredient may be in powder form for constitution with a suitable vehicle, such as sterile pyrogen-free water, before use.

The compounds may also be formulated in rectal compositions such as suppositories or retention enemas, e.g., containing conventional suppository bases such as cocoa butter or other glycerides.

In addition to the formulations described previously, the compounds may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

A pharmaceutical carrier for the hydrophobic compounds of the invention is a cosolvent system comprising benzyl alcohol, a nonpolar surfactant, a water-miscible organic polymer, and an aqueous phase.

The cosolvent system may be the VPD co-solvent system. VPD is a solution of 3% w/v benzyl alcohol, 8% w/v of the nonpolar surfactant polysorbate 80, and 65% w/v polyethylene glycol 300, made up to volume in absolute ethanol. The VPD co-solvent system (VPD:5W) consists of VPD diluted 1:1 with a 5% dextrose in water solution. This co-solvent system dissolves hydrophobic compounds well, and itself produces low toxicity upon systemic administration. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of polysorbate 80; the fraction size of polyethylene glycol may be varied; other biocompatible polymers may replace polyethylene glycol, e.g., polyvinyl pyrrolidone; and other sugars or polysaccharides may be substituted for dextrose.

Alternatively, other delivery systems for hydrophobic pharmaceutical compounds may be employed. Liposomes and emulsions are well known examples of delivery vehicles or carriers for hydrophobic drugs. Certain organic solvents such as dimethylsulfoxide also may be employed, although usually with a greater toxicity.

Additionally, the compounds may be delivered using a sustained-release system, such as semipermeable matrices of solid hydrophobic polymers containing the therapeutic agent. Various sustained-release materials have been established and are well known by those skilled in the art. Sustained-release capsules may, depending on their chemical nature, release the compounds for a few weeks up to over 100 days.

Depending on the chemical nature and the biological stability of the therapeutic reagent, additional strategies for protein stabilization may be employed.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Many of the compounds identified with the assays of the present invention may be provided as salts with pharmaceutically compatible counterions. Pharmaceutically compatible salts may be formed with many acids, including but not limited to hydrochloric, sulfuric, acetic, lactic, tartaric, malic, succinic, etc. Salts tend to be more soluble in aqueous or other protonic solvents that are the corresponding free base forms.

Gene sequences of TDNEs identified by means of the invention may be administered as gene therapy agents. The gene sequences encoding TDNEs identified and exerting the desired therapeutic effect can be introduced either in vivo or ex vivo into cells for expression in a mammalian subject. Genes sequences of TDNEs developed through the invention may also be administered by other known methods for introduction of nucleic acid into a cell or organism (including, without limitation, in the form of viral vectors or naked DNA). Cells may also be cultured ex vivo in the presence of proteins of the present invention in order to proliferate or to produce a desired effect on or activity in such cells. Treated cells can then be introduced in vivo for therapeutic purposes.

5.8.3. Effective Dosage

Pharmaceutical compositions suitable for use in the present invention include compositions wherein the active ingredients are contained in an effective amount to achieve its intended purpose. More specifically, a therapeutically effective amount means an amount effective to prevent development of or to alleviate the existing symptoms of the subject being treated. Determination of the effective amounts is well within the capability of those skilled in the art, especially in light of the detailed disclosure provided herein.

For any compound used in the method of the invention, the therapeutically effective dose can be estimated initially from cell culture assays. For example, a dose can be formulated in animal models to achieve a circulating concentration range that includes the IC₅₀ as determined in cell culture (i.e., the concentration of the test compound which achieves a half-maximal inhibition of the target protein's biological activity). Such information can be used to more accurately determine useful doses in humans.

A therapeutically effective dose refers to that amount of the compound that results in amelioration of symptoms or a prolongation of survival in a patient. Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio between LD₅₀ and ED₅₀. Compounds which exhibit high therapeutic indices are preferred.

The data obtained from these cell culture assays and animal studies can be used in formulating a range of dosage for use in human. The dosage of such compounds lies preferably within a range of circulating concentrations that include the ED₅₀ with little or no toxicity. The dosage may vary within this range depending upon the dosage form employed and the route of administration utilized. The exact formulation, route of administration and dosage can be chosen by the individual physician in view of the patient's condition. (See, e.g., Fingl et al., 1975, in “The Pharmacological Basis of Therapeutics”, Ch. 1 p1).

Dosage amount and interval may be adjusted individually to provide plasma levels of the active moiety which are sufficient to maintain the kinase modulating effects, or minimal effective concentration (MEC). The MEC will vary for each compound but can be estimated from in vitro data; e.g., the concentration necessary to achieve 50-90% inhibition of the kinase using the assays described herein. Dosages necessary to achieve the MEC will depend on individual characteristics and route of administration. However, HPLC assays or bioassays can be used to determine plasma concentrations.

Dosage intervals can also be determined using MEC value. Compounds should be administered using a regimen which maintains plasma levels above the MEC for 10-90% of the time, preferably between 30-90% and most preferably between 50-90%. In cases of local administration or selective uptake, the effective local concentration of the drug may not be related to plasma concentration.

The amount of composition administered will, of course, be dependent on the subject being treated, on the subject's weight, the severity of the affliction, the manner of administration and the judgment of the prescribing physician.

5.8.4. Packaging

The compositions may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration. Compositions comprising a compound of the invention formulated in a compatible pharmaceutical carrier may also be prepared, placed in an appropriate container, and labelled for treatment of an indicated condition. Suitable conditions indicated on the label may include inhibition of cell proliferation, treatment of a tumor, treatment of arthritis, and the like.

The following examples are given to enable those skilled in the art to more clearly understand and to practice the present invention. The present invention, however, is not limited in scope by the exemplified embodiments, which are intended as illustrations of single aspects of the invention only, and methods which are functionally equivalent are within the scope of the invention. Indeed, various modifications of the invention in addition to those described herein will become apparent to those skilled in the art from the foregoing description and accompanying drawings. Such modifications are intended to fall within the scope of the appended claims.

### 6. EXAMPLES

### 6.1. Example 1

### Identification And Isolation Of TDNE For Ras-Raf Interactions

The interactions of Ras and Raf have been well characterized and provide a model system to demonstrate the successful use of the methods and compositions of the invention. Recent reports show that a synthetic Raf-Raf interaction can promote constitutive Ras pathway signaling. Flory et al., 1998, J. Virol. 72:2788-2794; Mineo et al., 1997, J. Biol. Chem. 272:10345-10348. The following example describes successful use of a yeast-based modified dual-bait strategy and an AraC-based system to identify TDNEs that block ras-raf protein-protein interactions.

Introduction To Ras-Mediated Signal Transduction And Ras-Raf Interactions. Ras proteins are plasma membrane-bound GTPases that function as relay switches transducing extracellular signals to the nucleus. In normal cells, Ras proteins cycle between the inactive GDP- and active GTP-bound forms to regulate cell proliferation and differentiation. Details of the signal transduction cascade leading to the activation of transcription factors, like cFos, has been the subject of numerous review articles (e.g., McCormick, 1994, Curr. Opin. Genet. Dev. 4:71-76; Marrshall, 1996, Curr. Opin. Cell Biol. 8:197-204). In a large number of human cancers, Ras is locked in its GTP-bound form due to mutations in amino acids 12, 13 and 61 leading to constitutive signalling (Lim et al., 1996, Eur. J. Biochem. 242:171-185). As a result, the Ras pathway no longer requires an a upstream growth signal, and Ras down stream components such as c-Raf-1, MEK and MAPK are constitutively activated, which causes cell transforming phenotypes. The transforming phenotypes include lost of contact growth inhibition, growing in semi-solid mediun, e.g., soft agar, increased proliferation rate, and disorganized actin filaments. Dominant-negative mutants of Ras and Raf have been shown to block these transformed phenotypes which depend on the interaction of Ras with Raf

The ability to isolate successfully identify TDNEs, using the yeast-based modified dual-bait strategy of the invention provides an excellent demonstration of the successful use of the present invention. The TDNEs identified herein in a microbial system can then be tested for their ability to reverse the transformed phenotype of Ras-transformed cells.

Identification And Isolation of Interaction-Based Raf And Ras Blocking TDNEs In A Yeast System. The yeast-based modified dual bait system utilized herein includes a specific yeast background supporting a modified dual-bait two-hybrid dominant-negative scheme for the identification of TDNEs derived from human HA-Ras, human c-Raf1 and human activated Raf1.

In particular, this system for the identification of TDNEs from human HA-Ras comprises:

(1) a LexA-dfRas (df for mutagenized CAAX box) fusion gene integrated into the yeast SKY191 chromosome and placed under the control of the ADH promoter region;

(2) cI-operator-LacZ reporter fusion gene on a two (2) micron plasmid under the control of an activator dependent minimal Gal1 promoter region;

(3) Activation Domain-c-Raf1 fusion gene (AD-cRaf1) on a two (2) micron plasmid under the control of galactose inducible Gal1 promoter;

(4) LexA-operator-LacZ reporter fusion gene on a two (2) micron plasmid under the control of an activator dependent minimal Gal1 promoter region; and

(5) cI-TDNE fusion library on a two (2) micron multicopy plasmid under the control of the ADH promoter region.

When SKY-191 yeast cells with the integrated LexA-dfRas fusion gene and the activation domain-cRaf1 fusion gene on two (2) micron plasmid are grown on media with galactose, both fusion proteins are synthesized and form dimers. This dimer formation results in the activation of transcription of two reporter genes: the LexA-operator-Leu2 gene in the chromosome and the LexA-operator-LacZ reporter gene on the two (2) micron plasmid. As a result, yeast can grow on synthetic dropout medium without leucine and produce a blue color on medium containing XGal. When a library of potential TDNEs, i.e., cI-dfRas fusions, are over-expressed in this genetic background (from a two (2) micron plasmid) those cI-TDNE fusions proteins which efficiently interact with the AD-cRaf1 fusion result in the transcription activation of cI-operator-Lys2 reporter fusion. This activation allows yeast with the TDNE cI-dfRas fusions to be selected on dropout medium without lysine. A subset of the lysine-selected fusions may interact strongly with the AD-cRaf1 fusion and cause the complete loss (or modulation) of activation of the first two reporter genes, i.e., LexA-operator-Leu2 and LexA-operator-LacZ. Such fusions show both the “trap” and “blocking” phenotypes and are, therefore, candidate dominant-negative elements to be tested in tissue culture models for the reversal of the Ras transforming phenotype.

The Isolation Of Interaction-Based Raf blocking TDNE In The E. coli AraC system. Although AraC chimera have previously been described (Bustos and Schleif, 1993, Proc. Natl. Acad. Sci. USA 90:5638-5642; Soisson et al., 1997, Science 276:421-425), the strains and plasmids used in the application of AraC to the isolation of TDNE were developed herein.

Host Reporter System Strain And Construction Scheme. The E. coli host strain, SAD4, combines several chromosomal modifications to allow for efficient and sensitive detection of AraC_{DNA} dimerization. The wild-type E. coli araC must be inactivated to avoid competition between the engineered AraC_{DNA} fusion protein and AraC. The host strain must also contain a reporter gene that is expressed at elevated levels in the presence of dimerized AraC_{DNA} fusion proteins.

SAD4 was constructed by P1ᵥᵢᵣ transduction. The Tn10 marker from strain LJ2808 (F⁻,fruR11::Tn10) was crossed into strain M8834 (F⁻, Δ1109 (araC-leu), rpsL150) to produce strain SAD3. P1vir(SAD3) crossed into strain RH8669 (F⁻, thi, ΔU169 (lacIPOZYA argF), fla, relA rpsL araD139, araB::Mu_{cts}62/λp1, ΔW209 (trp-locO)) to produce strain SAD4. This strain has the requisite genotype (ΔaraC, araB::lacZ) for the reporter strain.

Construction of a positive and-negative control plasmid. To verify the ability of engineered AraC_{DNA} fusion protein to produce a detectable signal in this system, a set of control plasmids was constructed. The control plasmids encoded a polypeptide comprising a known dimerization domain, the leucine zipper domain from the yeast protein GCN4, fused to the araC_{DNA} domain. Plasmid pKM19-C170 was the source of the 'araC DNA binding domain. The truncated 'araC gene encodes residues 170-292. Leucine zipper dimerization domain was amplified from plasmid pGCN4-leu using primers DAE3 5′-CCC CTC GCA GTA TTA CTG CTC ACT AAC-3′ and DAE4 5′-ACG TTC ACC AAC TAG TTT TTT CAG G-3′. A nonfunctional leucine zipper was also amplified from plasmid pGCN4-pro using primers DAE3 and DAE4. Both PCR products were separately cloned into pKM19-C170 such that the fragment encoding the leucine zipper dimerization domain was in frame with the AraC_{DNA} encoding domain. The resulting plasmids, pWE1 and pWE2, contained the GCN4_{dimer}::AraC_{DNA} fusion protein under the control of the lac promoter. The lac promoter and GCN4_{dimer}::AraC_{DNA} region from plasmids pWE1 and pWE2 were amplified by PCR using primers

| | |
|---|---|
| DAE1 | 5′-CTG ATC CCC GGG ACC GAG CGC AGC GAG TCA G-3′, and |
| DAE2 | 5′-CTG ATC CCC GGG CTG CAA ACC CTA TGC TAC-3′ |

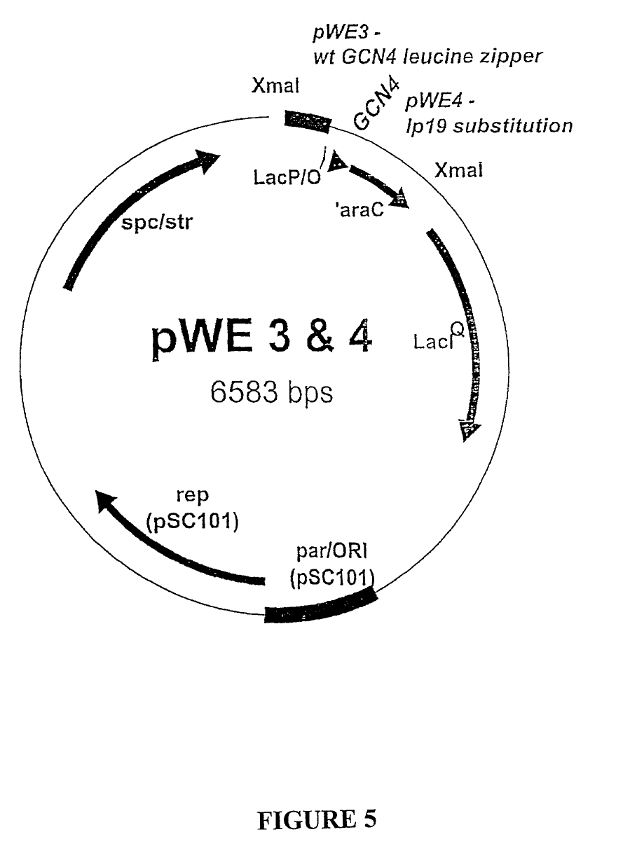

and cloned into plasmid pMS421 at the XmaI site. Plasmid pMS421 is a low copy vector and also contains the lacI gene, which encodes the lac repressor protein. Plasmids pWE3 and pWE4 are the pMS421 based plasmids containing either the wild-type GCN4_{dimer}::araC_{DNA} or the GCN4_{mut. dimer}::AraC_{DNA} constructs (FIG. 5). Only the wild-type GCN4 fusion activates the reporter gene, the mutant GCN4, that is defective in dimer formation does not. These activation values provide standards against which activation by other fused domains can be compared.

Vector For Screening In Frame Tagged Fragments. Tagged fragments were expressed in an E. coli strain that already expresses the Lac-regulated AraC fusion protein from apSC101 origin-based plasmid maintained under selection with spectinomycin. The vector pASK75 was used as a substrate for the expressing the TDNE candidates. It is based on the colE1 origin, is ampicillin selectable and includes the anhydrotetracycline-inducible tet promoter (Freundlieb et al., 1997, Methods Enzymol. 283:159-173) and is, thus, compatible with the AraC-expressing vector. The gene encoding the enhanced green fluorescent protein (EGFP) was cloned downstream of the tet promoter. Separating the tet promoter region from the EGFP gene is a region of DNA that contains multiple copies of a transcriptional terminator (T4 region). Excision of the T4 region followed by religation will produce a plasmid with the EGFP gene out of frame with respect to the tet promoter's associated translational start signal. Insertion of randomly fragmented gene segments, encoding subfragments of the dimerization polypeptide fused to the AraC-DNA binding domain, can restore the reading frame of the EGFP gene by translationally fusing such subfragment polypeptides to the EGFP gene. Expression of the fusion product is controlled by the tet promoter. Correct in frame fusions, candidates for TDNE, are identified by virtue of their anhydrotetracycline-inducible fluorescent phenotype. Appropriate clones are then examined for the TDNE phenotype by looking for the reversal of the AraC activation function (a Lac⁺ phenotype becoming Lac⁻.)

Raf Construction. The fall length raf gene was PCR amplified to contain PstI and BamHI ends using primers

| | |
|---|---|
| DAE5 | 5′-GAC ATA CTG CAG GAT GGA GCA CAT AC-3′, and |
| DAE6 | 5′-CTG TAT GGA TCC AGG AAG ACA GGC AG-3′. |

This PCR fragment was cloned into PstI and BamHI cut pKM19-C170 such that the raf gene product was in frame with the araC_{DNA} gene product. The entire fusion was PCR amplified to contain XmaI ends with primers DAE1 and DAE2, see, supra, and cloned into XmaI cut pMS421. Dimerization potential was evaluated by transforming the strain into SAD4 and measuring β-galactosidase levels.

The microbial approaches described above (modified dual-bait in yeast and AraC in E. coli) successfully identified a number of candidate TDNEs that inhibit ras-raf protein-protein interactions.

6.2. Example 2

### Evaluation of Candidate Ras and Raf TDNEs

The Example described, above, in Section 6.1, demonstrates the successful identification of candidate TDNEs that block ras-raf protein-protein interactions. Evaluation of their nature as functional TDNE requires expression in Ras-transformed cells and a determination of the effect TDNE has on such cells.

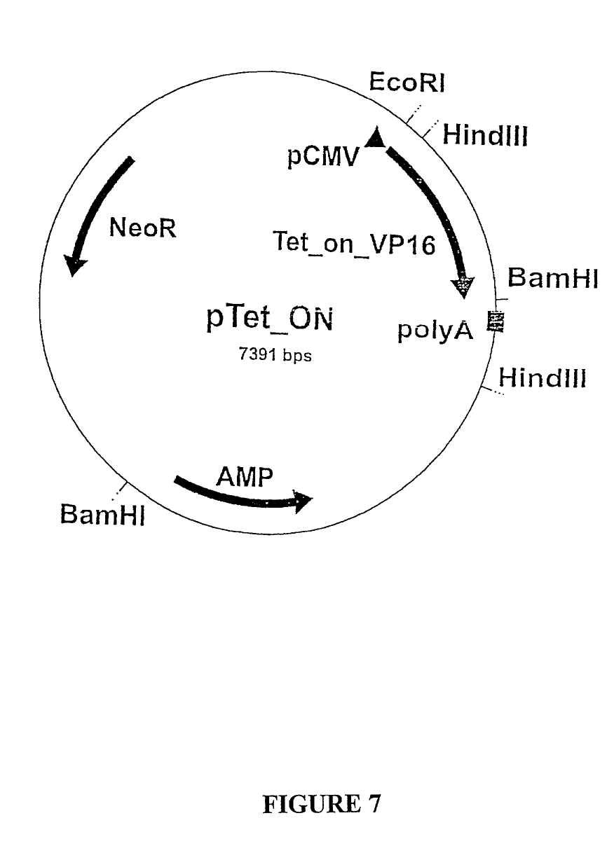

For example, identification of TDNEs that inhibit oncogenic Ras interaction with Raf, and hence interrupt the Ras signaling pathway and reduce cell transforming potential can be identified via such assays. An oncogenic Ras-transformed cell line with an inducible gene expression system can be established or utilized for such studies. For example, mammalian cells transformed by human oncogenic Ras gene are transfected with Tet-on expression system plasmids (Clontech, Palo Alto, Calif.; FIG. 7). The candidate Ras TDNE DNA fragments identified in microbial systems (see, e.g., Section 6.1) are inserted into the Tet-on plasmid pTRE, see, infra, in which the DNA fragment expression in the host cells is controlled by the availability of doxycycline (Dox) or tetracycline (Tc) in the cell growth media. The change of cell transforming potential by controlling the expression of Ras dominant-negative DNA fragments are evaluated by measuring cell proliferation rate, DNA synthesis, MAPK activity, cell growth in soft agar and c-Fos promoter activation.

Cell Culture. The mammalian cells lines that are oncogenic by virtue of being Ras-transformed are utilized herein. For example, either CHO or NIH 3T3 cells are transfected with human oncogenic Ras and the Tet-on expression system plasmids. Cells are maintained at 37° C. in Dulbecco's Modified Eagle's Medium (DMEM) with 10% fetal bovine serum (FBS), 2 mM L-glutamine, 100 units/ml Penicillin and 100 μg/ml streptomycin in a humidified chamber with 5-10% CO₂.

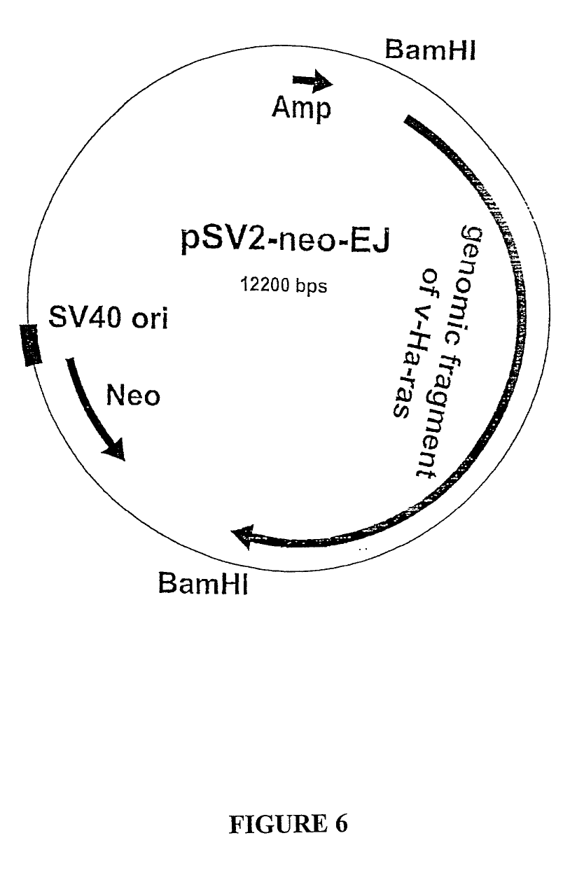

Plasmids. In mammalian cells, ras genes encode for a family of closely related 21-kDa proteins, including N-ras, K-ras and H-ras. Activated ras oncogenes have been identified in various forms of human cancer, including carcinomas of the lung, colon and pancreas. The ras gene used herein is an H-ras oncogene cloned from human bladder carcinoma cells with a mutation at codon 12 (glycine to valine). The 6.6 kb genomic DNA (FIG. 6) (Parada et al., 1982, Nature 297:474-478) is inserted into the BamHI site of vector plasmid pSV2-neo (Berg, 1982, J. Mol. Applied Genet. 1:327-341) to construct plasmid pSV2-neo-EJ (FIG. 6).

Establishing Inducible Gene-Expression Cell Lines. The oncogenic Ras and Tet-on expression system plasmids are introduced by liposome-mediated electroporation method. For stable transfection, 10 μg of plasmid DNAs in mammalian cell growth medium without serum and antibiotics, is first mixed with 5 μl of liposome (Lipofectin Reagent, 1 mg/ml, Bethesda Research Laboratories, Gaithersburg, Md.) and incubated at room temperature for 30 min. The plasmid/liposome mixture is then added to the recipient cell suspension (3×10⁶ cells) and electroporated at 180 V, 700 μF (BTX Electro Cell Manipulator 600, Genetronics, Inc., San Diego, Calif.). After electroporation, the cells are split into ten 60 mm Petri dishes with regular cell medium. On the second day, the cells are changed to cell medium with 0.5 mg/ml G418. The G418-resistant colonies are isolated two (2) to three (3) weeks after the onset of selection, and single colonies are established into independent cell lines. The G418-resistant cell lines are then tested for luciferase activity by following the manufacturer's protocol (Promega, Madison, Wis.). The cell line with the highest luciferase activity is selected for introducing Tc- or Dox-inducible plasmids by stable transfection as described above. The cells are selected by hygromycin. Hygromycin-resistant colonies are tested for the expression of interested dominant-negative DNA sequence with or without Tc or Dox in the cell growth media. The best colonies are selected based on functional assays. In one embodiment, the cells transfected with Ras-dominant-negative sequence are turned on or off by Tc or Dox, and then cell transforming potentials are tested.

Methods For Measuring Cell Transforming Potentials. To examine the efficacy of DNA dominant-negative sequence that interrupts protein-protein interaction in mammalian cells, specific functional assays are employed. In one embodiment, the Ras dominant-negative sequence is accessed by the cell phenotype changes in oncogenic Ras-transformed cells. Oncogenic Ras-transformed cells generally show increased cell proliferation and DNA synthesis rate, growing in semi-solid media, e.g., soft agar, sustained activation of e.g., AMPK and c-Fos.

DNA synthesis is measured by [³H] thymidine incorporation. CHO cells transfected with different dominant-negative inserts are plated in 24-well plates at 3×10⁴/well in growth medium at 37° C. for 24 hr. The cells are then washed three times with phosphate-buffered saline (PBS) and incubated in the growth medium supplemented with or without Dox for 24 hr. The cells are then incubated at growth medium containing 5 μCi/ml [³H] thymidine with or without Dox for 16 hr, and the radioactivity incorporated into the trichloroacetic acid-insoluble fraction is determined by liquid scintillation spectrometry.

For soft agar growth assays, CHO cells transfected with different dominant-negative inserts are plated in 6-well plates at a density of 1×1 per well (without Dox in the growth medium) or 1×10⁵ per well (containing Dox in the growth medium). The soft agar plates consist of 6 ml 0.5% Noble agar medium base layer with cells on the top layer which contains 1.5 ml of 0.3% Noble agar medium with or without the inducer Dox. Cells are incubated for 3 weeks at 37° C., and colonies larger than 0.1 mm in diameter are scored.

Cell growth rates are determined either by Trypan blue dye exclusion method or a cell proliferation kit available from Clontech (Palo Alto, Calif.).

To determine how the Ras dominant-negative sequences affect MAPK activity in oncogenic Ras-transformed cells, cells are grown in 100 mm tissue culture in the medium with or without inducer Dox for 48 hr. Cells are then washed with 10 ml PBS, and lysed in buffer (50 mM Tris-HCl (pH 8.0), 150 mM NaCl, 0.5 % deoxycholic acid, 1% NP-40, 10% glycerol, 50 mM NaF with 10 μg/ml leupeptin and aprotinin, 1 mM phenylmethylsulfonyl fluoride and 1 mM Na₃ VO₄) for 30 min on ice. 200 μg of cell lysate from each cell line are used for immunoprecipitation with 1 μg of anti-extracellular signal-regulated kinase 2 (ERK2) serum. The immunoprecipitates are then washed twice with the lysis buffer, twice with kinase wash buffer containing 50 mM Tris/HCl (pH 7.5), 10 mM MgCl₂ and 1 mM dithiothreitol (DTT) and once with kinase buffer which is kinase wash buffer containing 50 mM ATP. Each MAPK reaction mixture contains 30 μl of kinase buffer, 5 μl of [γ-³²P]-ATP (1 μCi/μl) and 2 μl of the substrate MBP (5 μg/μl). After incubating at 30° C. for 15 min, the reactions are stopped by adding 8 μl of 6× sample buffer and heating at 95° C. for 5 min. The sample are analyzed by 12% SDS polyacrylamide gel and exposed to X-ray film, or quantitated by liquid scintillation counting.

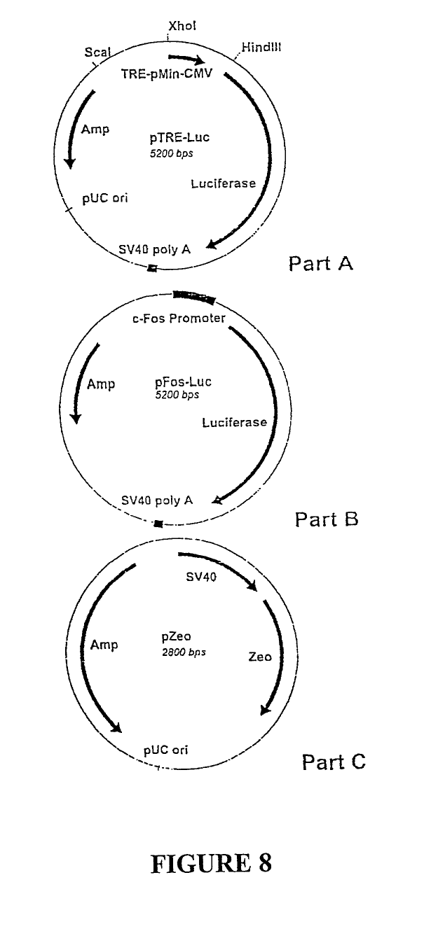

The c-Fos transcription factor is one of the earliest genes that are stimulated by mitogenic signals. Ras-induced mitogenic signals is believed to cause phosphorylation of c-Fos promoter-binding proteins, resulting in c-Fos expression. To test whether the Ras dominant-negative sequences that are identified from microbial systems can interrupt the Ras signaling in the oncogenic ras-transformed mammalian cells, the c-Fos expression is determined. For easy detection, the c-Fos promoter/enhance region positions −711 to +41, see, FIG. 8A) replaces the TRE region of pTRE-Luc plasmid to make pFos-Luc plasmid FIG. 8B. pFos-Luc plasmid is then co-transfected with a zeomycin-resistant plasmid FIG. 8C into the cells containing Ras dominant-negative plasmids. Zeomycin-resistant cells with highest luciferase activity (using Promega luciferase activity kit, Promega, Madison, Wis.) are selected. The effect of Ras dominant-negative sequence on c-Fos expression is determined by growing cells in the medium with or without inducer Dox, and comparing luciferase activity.

### 6.3. Example 3

### Analysis Of Competition Between Sequences For The Activation Domain-Partner Protein In The Modified Dual-Bait System

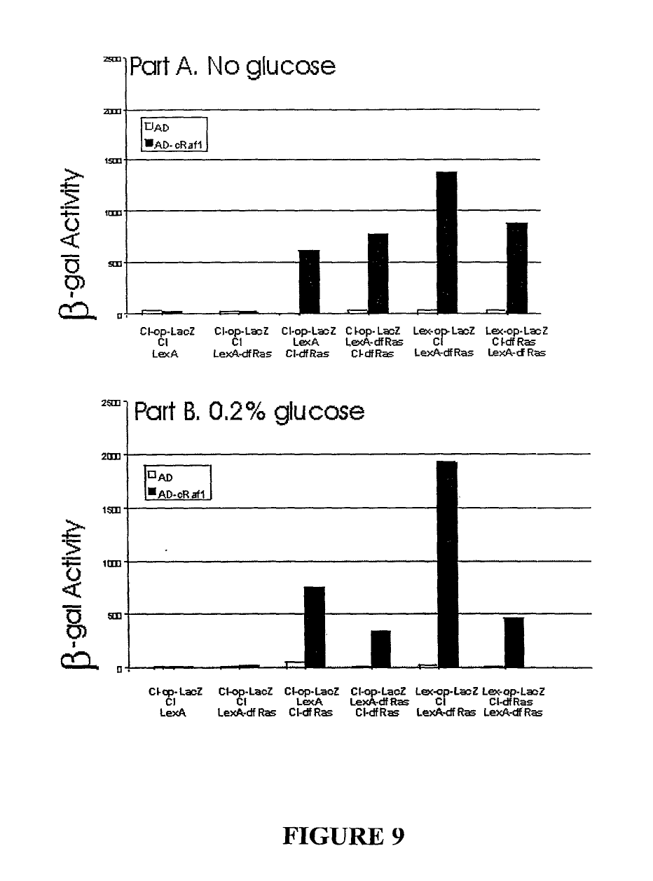

In this example, competition between LexA-dfRas and CI-dfRas for interaction with Ad-cRaf1 in yeast S. cerevisiae strain SKY48 is illustrated. In order to isolate TDNEs from a library, the full-length HA-Ras1(C186G) fused to the LexA DNA binding protein (DBP) and the TDNE candidates fused to CI-DBP must compete for binding to the AD-cRaf1 fusion protein. If the AD-cRaf1 fusion protein is present in excess, competition will not be observed. Under the conditions normally employed in yeast protein interaction assays (2% galactose, 1% raffinose), such competition is not seen (see FIG. 9A). In order to observe such a competition, the level of AD-cRaf1 fusion protein was down-regulated by systematically varying the concentration of glucose (an inhibitor of regulated gal promoter expression) in the growth media.

To construct the relevant series of strains, SKY48 was transformed with either CI or CI-dfRas followed by zeocin selection. A second set of transformations was then performed simultaneously introducing three plasmids containing either: the AD (alone) or AD-cRAF1, the second DNA binding domain partner LexA, either alone or fused with dfRas and one of the two reporter plasmids carrying either CI-operator-LacZ or LexA-operator-LacZ fusions. After the final round of transformations, 8 separate clones for each the 12 strains shown in FIG. 9 were isolated and inoculated in assay media. These strains were grown in minimal dropout media containing 2% galactose and 1% raffinose (FIG. 9A) to fully induce expression of the AD or (AD-cRaf1), or, alternatively, in the same media containing 2% galactose, 1% raffinose and 0.2% glucose (FIG. 9B) to partially induce expression of the AD (or Ad-cRaf1). Intermediate levels of glucose were also tested, to look at other levels of AD (or AD-cRaf) expression. The result shown in FIG. 9B demonstrated optimal competition between CI-dfRas and LexA-dfRas. Strains were grown for 6 hours in the indicated media and standard β-galactosidase assays were performed. FIG. 9 shows an average of results of the eight isolates that were assayed, with standard deviation of 10%.

The results indicate that the standard assay conditions fail to demonstrate significant competition between LexA-dfRas and CI-dfRas. The introduction of the competing LexA-dfRas fusion protein did not reduce the level of β-galactosidase expression with the CI-op-Lac reporter when CI-dfRas fused to the activation domain. Further, introduction of the competing C₁-dfRas fusion protein only slightly (35%) reduces the level of β-galactosidase expression with the LexA-op-Lac reporter, when LexA-dfRas is directing the activation domain. When the level of the expression of the activating domain is reduced (by the addition of 0.2% glucose) in the nonstandard assay, significant competition is detected. The LexA-dfRas fusion protein (with 0.2% glucose) reduces the level of β-galactosidase expression with the CI-op-Lac reporter when CI-op-Lac reporter when CI-dfRas is directing the activation domain. A 2-fold reduction is found where previously no reduction was seen. Further, a greater than 5-fold reduction is seen with the competing CI-dfRas fusion protein with 0.2% glucose repression where only a 35% reduction was noted in the standard assay with the LexA-op-Lac reporter, when LexA-dfRas is directing the activation domain.

### 6.4. Example 4

### Screening a Library of Ras Fragments for Members That Interact with Raf and Block the Ras-Raf Interaction Signal

The Example presented herein describes the successful generation and screening of a TDNE library of ras protein fragments. In particular, it is shown that through the use of the modified dual-bait system of the invention, an interaction signal could be titrated by co-expression of one of the interacting partners: the signal resulting from the LexA-Ras::Raf-AD interaction could be titrated by co-expression of the CI-Ras. A system in which an interaction signal can be attenuated by the co-expression one of the full length interaction partners (not fused to the signal generating assembly) is an important prerequisite to searching for fragments of that partner which can reduce the interaction signal.

It is noted that the results shown herein validate the TDNE approach of the present invention in that the TDNE identified in this screen for TDNEs that modulate ras-raf function contains a ras fragment that extends across a region of ras previously shown to be central to the ras-raf interaction.

It was demonstrated in Section 6.3, above, that standard implementations of the yeast 2-hybrid system fail to yield a 2-hybrid interaction signal that is subject to competition. Modification of this system, through both genetic (copy number) manipulation and gene regulation paradigms, allowed for the down regulation of the activation domain partner so that competition was observable.

In addition to a having an interaction signal subject to titration, application of this strategy entails fragmentation of the DNA target into smaller “domain sized” fragments for production of a TDNE library.

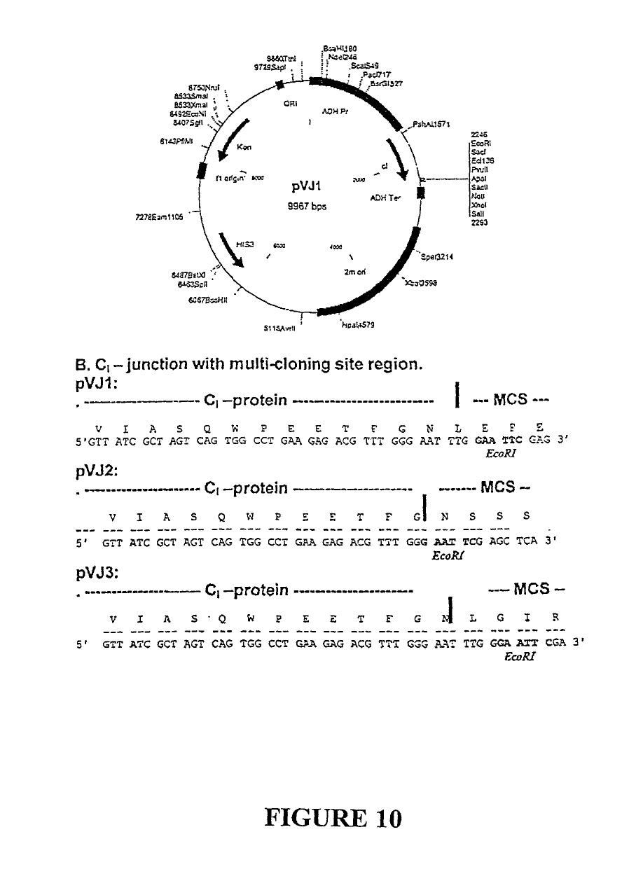

A fragment library based on the Ras protein, 300 ng of DNA from the plasmid pJD4-5-dfRas was created by partially digesting it with CviJI under relaxed condition. The restriction nuclease CviJI normally cuts DNA between RG and CY, but under relaxed conditions (10 mM Tris-HCl pH8.0, 10 mM NaCl, 20% DMSO, 20 mM DTT and 1 mM ATP) will cut DNA between PuG and CPy, PuG and CPu, and PyG and CpPy, approaching a cut every 16 nucleotides. DNA so cut is left with a blunt end, obviating the requirement for enzymatic treatment prior to a subsequent cloning step. Since the CviJI nuclease sites will be randomly placed with respect to the reading frame of the target protein (Ras in this example), a given fragment will only have a ⅓ chance of being cloned as an in frame fusion with the target carrier protein (CI, in the modified Dual-Bait 2-hybrid system). To ensure the potential for in frame fusions, regardless of the frame determined by the inserted fragments, three derivatives of the CI fusion vector (one for each reading frame) were constructed from a pGKS6 derivative (Serebriishii, I., Khasak, V., and Golemis, E A., 1999, J. Bio. Chem. 274: 17080-17087) by the insertion of synthesized nucleotides according to standard techniques (see FIG. 10 for details of the resultant plasmids). A range of partial digest levels were examined by varying the nuclease amount and conditions showing a full range of partial products were chosen. An array of fragments greater than about 30 base pairs was gel purified, concentrated, and ligated according to standard procedures. 300 ng purified fragments were ligated with 30 ng of Ec136 cut and dephosphorylated pVJ1,2,3 vectors; the vector was an equimolar mixture of the 3 different reading frame vectors, as described above. This mixture was transformed into the host E. coli strain DH5α and transformants were selected for on kanamycin (30 μg/ml) solid Luria broth plates at 37° C. About 4,500 colonies were pooled and amplified in liquid media (5 hours) and plasmid DNA (the pVJ-library) was purified with a Wizard Midi prep kit according to the vendor's recommendations (Promega; Madison, Wis.). Six μms of the pVJ-library were transformed into the yeast strain SKY48 (supra) selecting growth on solid agar with lysine in galactose-Raffinose media supplemented with zeocin. The vector alone (no peptide fused to CI) and the CI vector fused to full length Ras were used as negative and positive controls respectively.

After 3 days of incubation at 30° C., 545 Lys⁺ colonies appeared on the test plates. These colonies were examined for activation of the CI-op-LacZ reporter gene expression according to established procedures (Serebriiskii, I., Khasak, V., and Golemis, E A., 1999, J. Bio. Chem. 274: 17080-17087). All these colonies produced the same level of Lac expression whether grown on glucose (no activating Raf fusion produced) or Raffinose/galactose (activating Raf fusion induced) indicating that only self-activating CI-fusions had been isolated. After 5 days of incubation an additional 1500 colonies appeared and 360 were purified and tested for self-activation. Among these 360 colonies, 20 demonstrated Raf-dependent activation (Lys⁺ and Lac⁺ noted only on galactose/Raffinose media). These 20 selected clones were purified and plasmid inserts were characterized by PCR analysis using primers flanking the insert site,

5′-ATGATCCCATGCAATGAGAG-3′ (forward primer for cI-junction) and
5′-TTCGCCCGGAATTAGCTTGG-3′ (reverse primer).

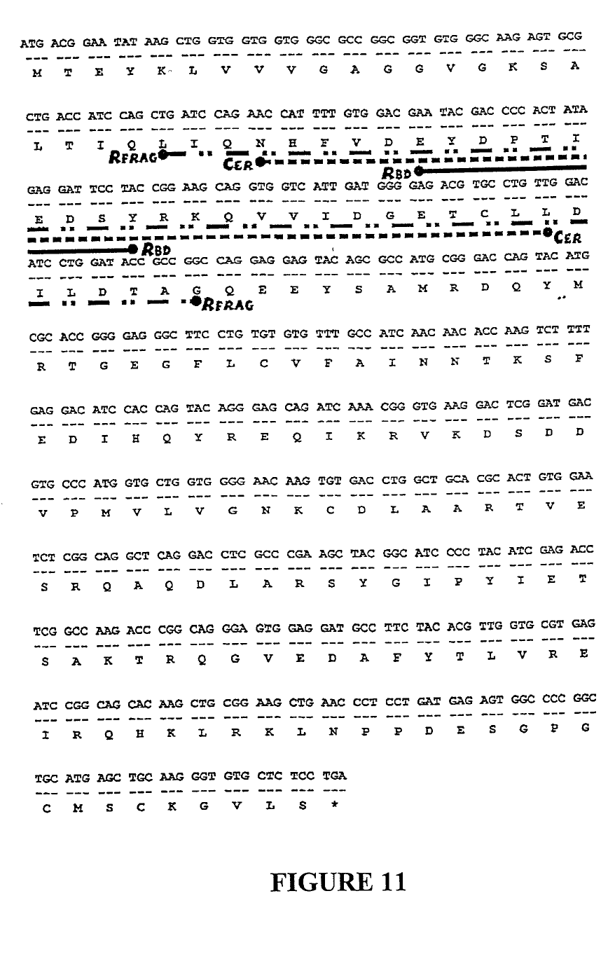

The PCR products indicated that 4 classes of clone sizes were present. DNA sequence was determined (using the PCR primers) for a few representative members of each class according the standard techniques. The most frequent class contained the Ras sequence highlighted (Rfrag) in FIG. 11 fused in-frame with the CI DNA binding protein. The other three classes of clones proved surprisingly to contain anti-sense segments of the Ras gene. All these anti-sense inserts were also in-frame with the CI product and contained a common (anti-sense) CviJI fragment. It is hypothesized that the common anti-sense fragment represents a fortuitous Raf binding peptide.

The Ras piece identified by this procedure is appropriate given what is known about the Ras-Raf interaction. FIG. 11 indicates the Ras segment identified in this study (Rfrag), a segment identified as being central to the Ras-Raf interaction interface (RBD; Marshall M., 1995, Mol Reprod 42:493-9; Terada T., et al., 1999, J Mol Biol 286:219-32), and a Ras fragment previously shown to bind Raf (CER, Fujita-Yoshigaki, J., et al., 1995, J. Biol. Chem. 270:4661-7 and references there in). This procedure identified the appropriate CviJ1 fragment extending across a region of Ras previously shown to be central to Ras-Raf interaction.

### 6.5. Example 5:

### Demonstration That an CadC-based Interaction Signal Is Subject to Titration by a Second Co-expressed Interacting Partner

This example demonstrates the successful identification of a TDNE using a CadC-based E. coli dimerization detection system, and shows that the signal generated in the CadC-based system is subject to competition, an important prerequisite for searching and identifying TDNEs containing fragments of a protein involved in a particular protein-protein interaction of interest.

In order to demonstrate that an EDDS interaction signal is subject to competition. The CadC truncation 1 (see FIG. 2, WO 99/23116) was used as a junction point for the formation of a CadC-TNFα chimera.

To obtain the TNFα sequences, appropriate PCR primers,

5′-TCTCCCCTGGAAAGGACACCATGAGC-3′ and
5′-GGCGTTTGGGAAGGTTGGATGTTCG-3′

were used to amplify TNFα from a human placenta Marathon-ready cDNA library (Clontech, Palo Alto, Calif.) using the Advantage-HF PCR kit (Clontech) according to the vendor's recommendations. The PCR fragments were cloned into the pGEM-T vector and transformed into the host strain JM109 (Promega, Madison, Wis.). Fidelity of the product was verified by DNA sequence analysis according to standard procedures and agreed with the published TNFα sequence (Marmenout, A., et al., 1985, Eur. J Biochem. 152, 515-522).

To create the CadC-TNFα chimera, TNFα was isolated (from the above pGEM-T clone) using the following PCR primers:

| | |
|---|---|
| 5′-AAGTCTGTCGACAGTCAGATCATCTTCTCTCG | (SalI site 5′ end) and |
| 5′-GCGGGATCCTCACAGGGCAATGATCCCAA | (BamHI 5′ end) |

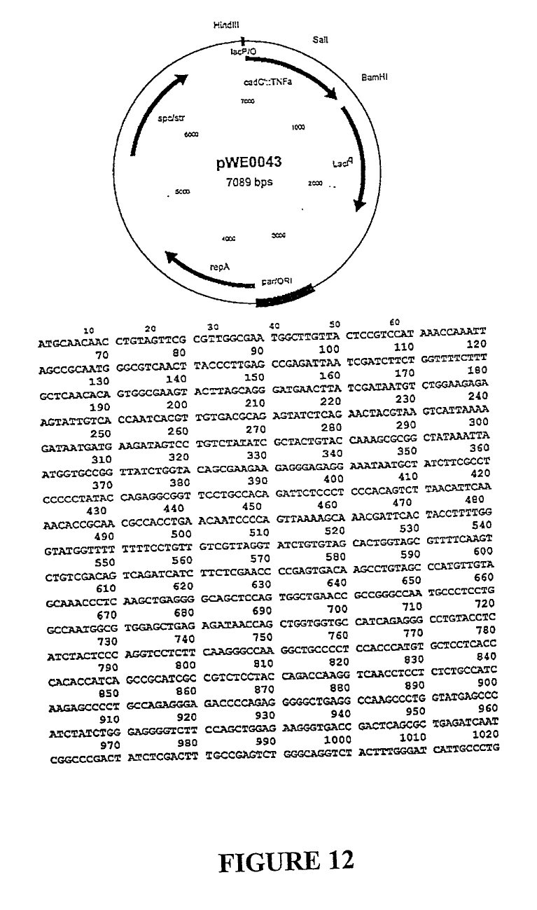

The desired CadC chimera was produced by the excision of the TNFαR ECD from the plasmid pCCT-I (see patent application EDDS) by digestion with SalI and BamHI, and replacment with the SalI and BamHI piece produced by digesting the PCR fragment of the TNFα extracellular domain employing standard molecular biology techniques (supra). The DNA sequence of the chimera in the resulting plasmid (pWE43) was determined to confirm integrity. The restriction map of pWE43 and the sequence of the CadC-TNFα chimera are given in FIG. 12.

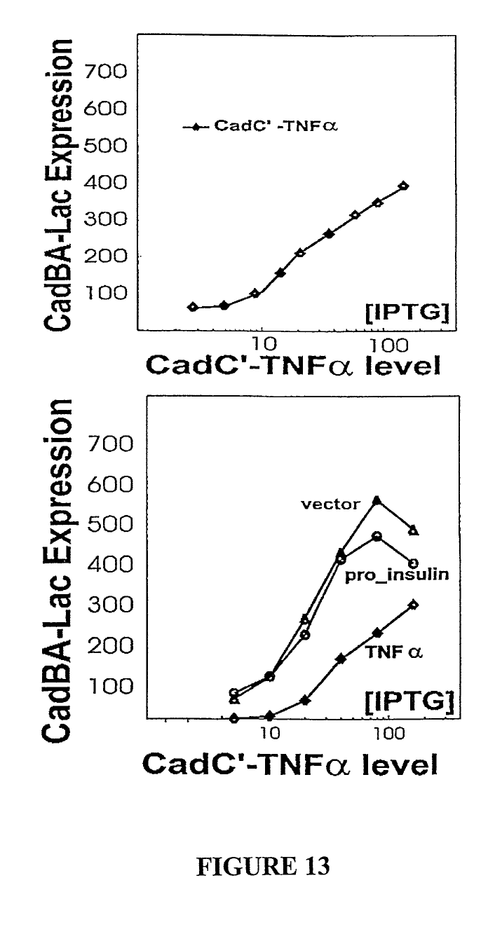

The plasmid pWE43 was transformed into E2088 (see WO 99/23116) and transformants were purified. Transformants were picked to individual wells of a microtiter plate and grown in 200 μl Luria Broth plus 25 μg/ml spectinomycin (without shaking) at 30° C. for 14 hours. At the end of the initial growth period cells were diluted back (1:40) and grown at the indicated IPTG concentrations for 5 hours. Cell turbidity was measured at 600 nm, the cells were made permeable by chloroform treatment, and aliquots were assayed for β-galactosidase activity using the substrate chlorophenol red β-D-galactoside (CPRG, Eustice, et al, 1991, Biotechniques 11: 739-742), as described by Menzel (Menzel R., 1990, Anal. Biochem 181: 40-50; WO 99/23116). The data in FIG. 13 (upper panel) demonstrated that induction of the CadC-TNFα construct resulted in the transcription of CadBA as monitored by β-galactosidase activity.

To see if this signal was subject to titration, it was necessary to co-express TNFα (and controls) together with the CadC-TNFα construct. In order to accomplish this, a second plasmid was designed to be compatible with the CadC-expressing pCCT-I and target the expressed protein to the periplasm. This vector was based on a colE1 origin of replication and an ampicillin selectable marker, and is thus compatible with the pSC101 origin and the spectinomycin selectable CadC vector(s). The periplasmic expression vector pBADa (WO 99/23116) was created by insertion of the OmpA signal sequence between the EcoR1 and Xba1 site of pBAD18 (Guzman et al., 1995, J. Bacteriol. 177:4121-4130). In addition to constructing this basic vector the cited patent also describes the cloning of the TNFα cytokine and proinsulin into the periplamsic expression vector. This set of plasmids provided the reagents required to test the CadC system for competition.

Examination of competition for the CadC-TNFα signal was accomplished by transforming the plasmids pBAD18 (vector), pASI (the proinsulin expressing plasmid), and pAST (the TNFα expressing plasmid), together with pWE43 [inducible CadC-TNFα], into the strain E2088 employing standard procedures and simultaneously selecting with ampicillin (100 μg/ml) and spectinomycin (25 μg/ml) on solid Luria Broth agar. Cultures of these 3 strains (200 μl in microtiter plates) were grown overnight in liquid Luria Broth (without shaking) at 30° C. with 100 μg/ml ampicillin and 25 μg/ml spectinomycin. At the end of the initial growth period the cells were diluted back (1:40) and grown at the indicated IPTG concentrations for 5 hours. Assays were performed as described above and results are shown in FIG. 13 (lower panel). The results indicated that the TNFα signal in the CadC chimera was subject to competition by co-expressed TNFα but not by the non-cognate protein proinsulin. Further, the competition was most pronounced (up to 50×) at low levels of chimera expression (low IPTG) as expected for a fixed level of competitor driven by basal ara promoter expression.

A variety of approaches were undertaken to isolate fragments of TNFα that could compete when expressed as OmpA_{leader} fusions. Competition was not observed with any of these pieces on their own. It was reasoned that a fusion of one or more of the fragments to a tag, e.g., a carrier protein might aid in either stabilizing the fragments and/or providing additional steric “bulk” that would aid in blocking the interaction. Fusions to the carrier protein MalE were constructed to test this hypothesis. MalE has been used successfully to direct the expression of many proteins to the periplasm of E. coli (Yanisch-Perron, C., et al., 1985, Gene 33, 103-119; Guan, C., et al., 1987, Gene 67, 21-30) and provides a convenient method of affinity purifying any “blocking” fusions of interest (Kellerman, O. K. and Ferenci, T., 1982, Methods in Enzymol. 90, 459-463; LaVallie, E., in Ausebel, F. M. et al. (eds), Current Protocols in Molecular Biology Greene Associates/Wiley Interscience, New York pp. 16.4.1-16.4.17).

An appropriate MalE fusion protein vector, compatible with the CadC system, was constructed by amplifying the malE gene from the vector pMAL-p2K (New England Biolabs, Beverly, Mass.) using the following PCR primers:

5′-GGAATTCAGGAGGAATTAACCATGAAAATAAAAACAGGTGCACGCATC-3′; EcoRI site and a synthetic RBS; and

5′-GCGGTGGAGCTCTACGTAAGTCTGCGCGTCTTTCAGGGCTTCATCGACA-3′ GTCTG; SnaBI and SacI sites.

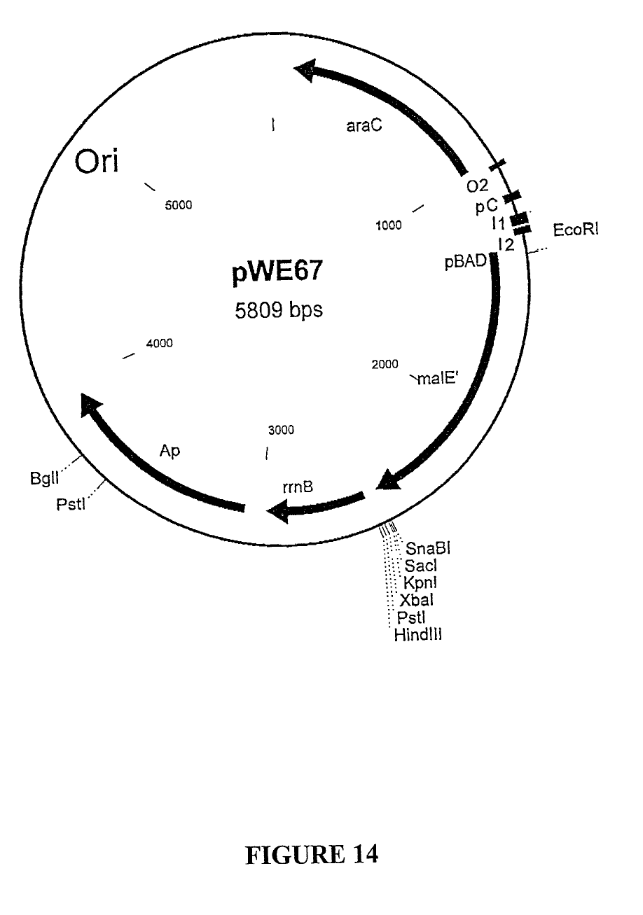

The PCR product was digested with EcoRl and Sacd and cloned into Sacd and EcoR1 digested pBAD18 (supra) using standard molecular biology techniques to create the plasmid pWE67 with the restriction map shown in FIG. 14. This vector is compatible with the CadC system (pWE67 has a ColE1 ori and a β-lactamase selection) and the SnaB1-HindIII cluster of restriction sites provides a means of generating fusions with the MalE protein.

Using pWE67 as an expression vector, a variety of fragments were found to compete to various extents. These same fragments when expressed without a tag, however, failed to show any competition, suggesting that a tag, in this case a carrier protein, is necessary to either stabilize the blocking fragments and/or to add steric “bulk”.

### 6.6. Example 6

### Isolation of Both Competing and Non-Competing Fragments with TNFαR

The extracellular domain of the TNFαR is an extended array with well defined subdomains which could provide a ideal test of the present invention to experimentally define appropriate fragments of a protein that might act as dominant genetic elements. The TNFαR receptor family is characterized by a modular organization of cysteine-rich subdomains that result in an elongated extracellular domain (Naisymth J H. and Sprang S R., TIBS 23 (1998) 74-79). An X-ray structure of the type I (55K) TNFαR extracellular domain complexed with the TNFα ligand has been published (Banner at el. Cell 7 (1993) 431-54) showing an apical bound trimeric ligand. It was previously shown that a CadC-TNFαR (typeII) chimera was able to support a robust interaction signal in the CadC system (see Section 6.5, above). To examine the ability of various segments of the receptor ECD to block the CadC-TNFαR interaction signal, fusions of various TNFαR-TDNE sequences were generated by fusing TNFαR ECD-encoding segments to sequences encoding MalE tags in the vector described above according to the procedures given below.

The following TNFαR ECD fragments were amplified employing the primers listed:

C77-T172

| | |
|---|---|
| C77-T172 | |
| 5′-GCTCTAGATGCACAGTGGACCGGGACACCGTG; | XbaI primer, and |
| 5′-AGGAGAAAGCTTTCATGTGGTGCCTGAGTCCTCAGT; | HindIII primer |
| D1-V84 | |
| 5-GCTCTAGAGATAGTGTGTGTCCCCAAGGAA; | XbaI primer, and |
| 5-TCCTCTAAGCTTTCACACGGTGTCCCGGTCCACTGTGCA; | HindIII primer. |
| D1-L154 | |
| 5-GCTCTAGAGATAGTGTGTGTCCCCAAGGAA; | XbaI primer, and |
| 5-TCCTCTAAGCTTTCACAACTTCGTGCACTCCAGGCTTTT; | HindIII primer. |

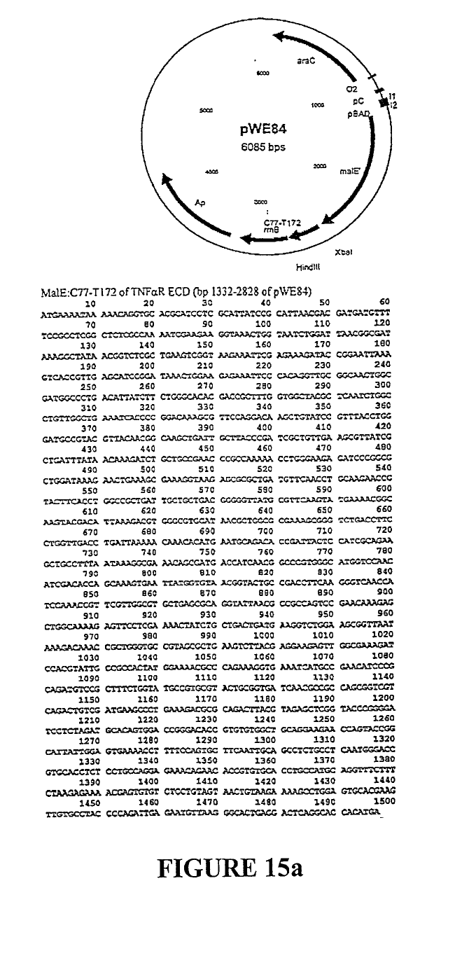

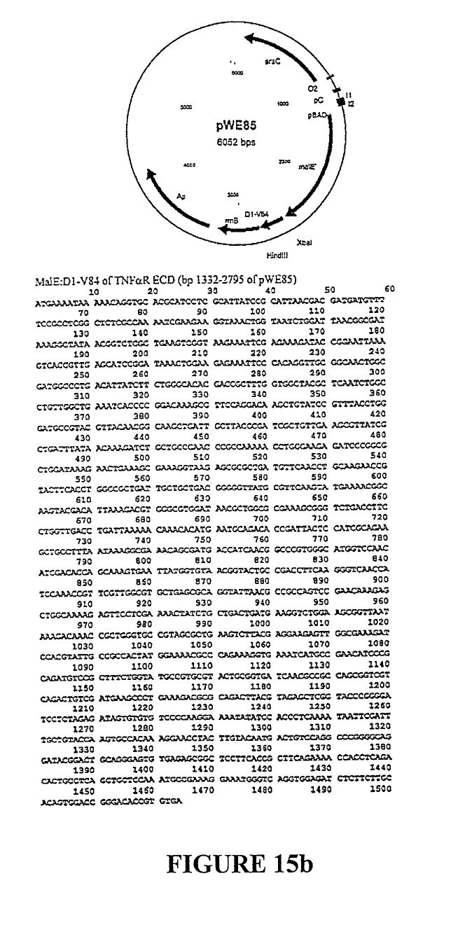

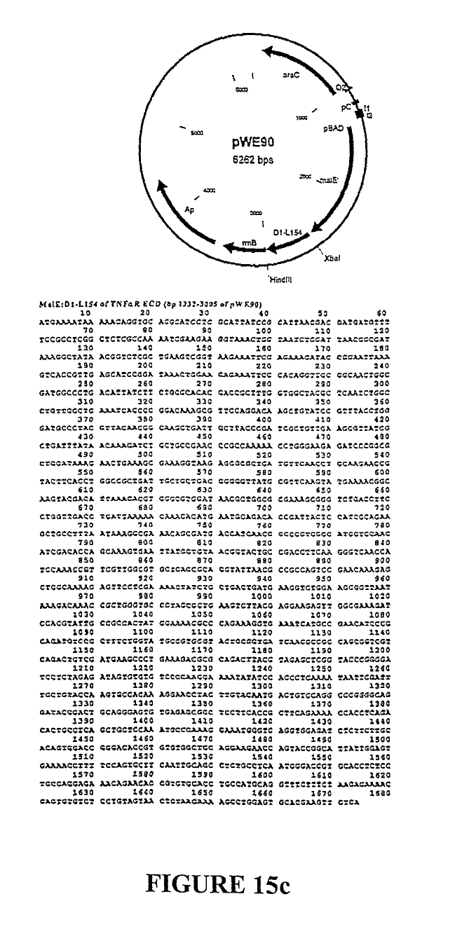

The amplified products were digested with XbaI and HindIII and cloned into XbaI-HindIII digested pWE67 to produce MalE fusion plasmids pWE84 (MalE::TNFαR segment C77-T172), pWE85(MalE::TNFαR segment D1-V84), and pWE90 (MalE::TNFαR segment D1-L154). DNA sequence analysis was performed to verify the integrity of the MalE fusions. A restriction map and sequence of these plasmids is given in FIGS. 15 A, B and C.

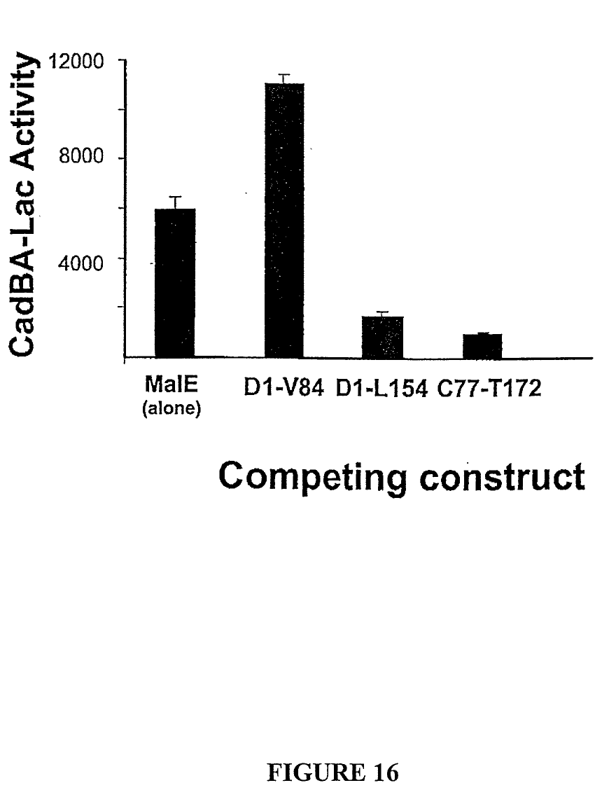

To examine competition of the CadC-TNFαR signal, the plasmids pWE67 (vector; MalE alone), pWE84, pWE85, and pWE90 were each individually transformed together with pCCT-1 (inducible CadC-TNFαR, see WO 99/23116) into strain E2088 employing standard DNA transformation procedures by simultaneously selecting ampicillin (100 μg/ml) and spectinomycin (25 μg/ml) on solid Luria Broth agar. Cultures (200 μl in a microtiter plate) of these 4 strains were grown together overnight in liquid Luria Broth with 100 μg/ml ampicillin and 25 μg/ml spectinomycin at 30° C. (without shaking). At the end of the initial growth period cells were diluted back (1:20,000) and grown overnight in LB plus 80 μM IPTG. Assays were performed as described in Example 5 and results are shown in FIG. 16.

The results show that the amino terminal segment of the TNFα receptor (residues D1-V84) fails to compete, while a longer piece (D1-L154) does. A segment with more carboxyl proximal sequence, but containing a deletion in amino terminal segment (C77-T172) shows marginally better competition. The level of CadC-TNFαR transcriptional activity noted for the MalE fusion protein was comparable to that seen for a strain without the co-expressed MalE protein, and the full length TNFαR ECD fused to MalE competed at comparable levels as that seen with the DI-L154 fragment (data not shown). Specificity is indicated by the observation that not all pieces derived from the TNF(XR ECD fused to MalE demonstrated competition.

The X-Ray crystal-derived structure for the 55 Kd form of TNFαR bound to the trimeric TNFα cytokine indicated that amino terminal residues are involved in receptor-cytokine interactions and that more membrane proximal segments of the receptor are involved in receptor-receptor interactions. The fact that the apical D1-84 fragment of the (75K form) TNFαR fails to show competition is consistent with this piece not being involved in receptor-receptor interactions.

The present invention is not to be limited in scope by the specific embodiments described herein, which are intended as single illustrations of individual aspects of the invention, and functionally equivalent methods and components are within the scope of the invention. Indeed, various modifications of the invention, in addition to those shown and described herein will become apparent to those skilled in the art from the foregoing description and accompanying drawings. Such modifications are intended to fall within the scope of the appended claims. Throughout this application various references are cited, the contents of each of which is hereby incorporated by reference into the present application in its entirety for all purposes.

What is claimed is:

1. A method for identifying a tagged dominant-negative element (TDNE) that interferes with interaction between a target protein and a partner protein comprising:
(a) expressing a TDNE in a microbial cell comprising a target protein and a partner protein wherein interaction between the target protein and the partner protein results in reporter gene expression, said TDNE comprising a fusion protein containing a portion of the target protein;
(b) measuring reporter gene expression; and
(c) comparing the level of reporter gene expression in (b) to the level obtained in the absence of the TDNE,
such that if the level in (b) is lower than that obtained in the absence of the TDNE, a TDNE that interferes with interaction between the target protein and the partner protein is identified.

2. The method of claim 1, wherein the portion of the target protein has a length of about six (6) amino acids to about 150 amino acids.

3. The method of claim 1, wherein the portion of the target protein has a length of about six (6) amino acids to about 30 amino acids.

4. The method of claim 1, wherein the target protein and the partner protein are each operably attached to a CadC domain.

5. The method of claim 1, wherein the target protein is operably attached to a DNA binding domain.

6. The method of claim 5, wherein the DNA binding domain is a CI domain.

7. The method of claim 5, wherein said DNA binding domain is a Gal1 domain.

8. The method of claim 5, wherein said DNA binding domain is an ADH domain.

9. The method of claim 1, wherein the target protein is operably attached to a DNA binding domain and the partner protein is operably attached to a transcriptional activation domain.

10. The method of claim 9, wherein the DNA binding domain is a LexA domain.

11. The method of claim 1, wherein the target protein and the partner protein are each operably attached to an AraC domain.

12. The method of claim 1, wherein the reporter gene is Leu2.

13. The method of claim 1, wherein the reporter gene is LacZ.

14. The method of claim 1, wherein the reporter gene is Lys2.
